(19) United States
(12) Patent Application Publication
(10) Pub. No.: US 2017/0239013 A1
(43) Pub. Date: Aug. 24, 2017

(54) PATIENT REFERENCE ASSEMBLY FOR AN ELECTROMAGNETIC NAVIGATION SYSTEM

(71) Applicant: General Electric Company, Schenectady, NY (US)

(72) Inventors: Dan Stephen Frame, Salt Lake City, UT (US); Daniel Eduardo Groszmann, Belmont, MA (US); Laurent Jacques Node-Langlois, Salt Lake City, UT (US); Michael Peter Orthner, Bountiful, UT (US)

(21) Appl. No.: 15/052,724

(22) Filed: Feb. 24, 2016

(51) Int. Cl. A61B 90/00; A61B 34/20

(52) U.S. Cl. CPC A61B 90/37; A61B 90/36; A61B 34/20; A61B 2090/376; A61B 2034/2051

(57) ABSTRACT

Methods and systems are provided for a patient reference assembly for an electromagnetic tracking system for use in image-guided surgery. In one embodiment, a patient reference assembly for an electromagnetic surgical navigation system includes a mounting platform including a first mating interface and an attachment interface shaped to couple to a patient. The navigation system further includes a sensor including a second mating interface shaped to removably couple with the first mating interface

### FIELD

Embodiments of the subject matter disclosed herein relate to an electromagnetic tracking system, and more particularly, to an electromagnetic tracking system for use in image-guided surgery.

### BACKGROUND

Electromagnetic tracking systems have been used in various industries such as aviation, motion sensing, retail, and medicine to provide position and orientation information for objects. They employ electromagnetic coils as electromagnetic transmitters and receivers. The electromagnetic field generated by the transmitter may be sensed by the receiver and used to estimate a position and/or orientation of the receiver relative to the transmitter.

In medical applications, electromagnetic tracking systems have proven particularly useful because they can track medical instruments such as catheters and needle tips within a patient's body, without line-of-sight requirements. Thus, when a medical instrument is obscured from view, such as when it is inserted into a patient's body, its position and/or orientation can still be obtained and visualized via the electromagnetic tracking system. An operator (e.g., a physician, surgeon, or other medical practitioner) may therefore more precisely and rapidly adjust the position of the medical instrument within the patient's body during image-guided surgery.

The medical instruments used during image-guided surgery may be equipped with a first electromagnetic coil assembly having a first electromagnetic coil, while a patient reference assembly may include a second electromagnetic coil (and thus may be referred to herein as a second electromagnetic coil assembly. In some examples, the patient reference assembly may be coupled to the patient anatomy to serve as a reference point for the electromagnetic tracking system. An electrical current may be supplied to either the first or second coil assemblies, generating an electromagnetic field. The electromagnetic field may in turn cause changes in the outputs from the two coil assemblies due to the mutual inductance between the coil assemblies. The position and/or orientation of the medical instrument may then be estimated based on changes in the outputs of the two coil assemblies. Together, the two coil assemblies may therefore provide an image of the instrument location relative to the patient anatomy to the operator.

Due to the engagement forces required to couple the patient reference assembly to the patient's anatomy, additional installation tools may be necessary to secure the patient reference assembly to the patient anatomy, thereby increasing the complexity of and time for system setup. Once the patient reference assembly is secured, its electrical cable may be obstructive to the user during surgery. However, it may not be possible to reposition the patient reference assembly during a surgical procedure due to having re-mount the patient reference assembly to the patient and having to recalibrate the system. Further, patient reference assemblies may be bulky and easily bumped, thereby causing a user to have to re-register the image produced by the navigation system. Further still, depending on where the patient reference assembly is attached to the patient anatomy, the shape and design of the patient reference assembly may need to be modified. Such constraints may require multiple different patient reference assembly designs which increases manufacturing costs.

### BRIEF DESCRIPTION

In one embodiment, a patient reference assembly for an electromagnetic surgical navigation system comprises a mounting platform including a first mating interface and an attachment interface shaped to couple to a patient and a sensor (e.g., patient reference sensor) including a second mating interface shaped to removably couple with the first mating interface. In this way, the sensor may be easily removed from the mounting interface and repositioned by user.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example image-guided surgery system that may include an electromagnetic tracking system according to an embodiment of the invention.

FIG. 2A shows a schematic of a first example of an electromagnetic tracking system that may be included in the image-guided surgery system of FIG. 1 according to an embodiment of the invention.

FIG. 2B shows a schematic of a second example of the electromagnetic tracking system of FIG. 2A according to an embodiment of the invention.

FIG. 3 shows a flow chart of an example method for tracking a medical instrument during an image-guided surgery using an electromagnetic tracking system according to an embodiment of the invention.

FIG. 4 shows a patient reference assembly coupled to anatomy of a patient in a first orientation according to an embodiment of the invention.

FIG. 5 shows a patient reference assembly coupled to anatomy of a patient in a second orientation according to an embodiment of the invention.

FIG. 6 shows an isometric view of a first embodiment of a patient reference assembly according to an embodiment of the invention.

FIG. 7 shows an isometric top view of a mounting platform of the first embodiment of the patient reference assembly shown in FIG. 6 according to an embodiment of the invention.

FIG. 8 shows a bottom view of a patient reference sensor of the first embodiment of the patient reference assembly shown in FIG. 6 according to an embodiment of the invention.

FIG. 9 shows a cross-sectional view of the first embodiment of the patient reference assembly shown in FIG. 6 according to an embodiment of the invention.

FIG. 10 shows an isometric top view of a mounting platform of a second embodiment of a patient reference assembly according to an embodiment of the invention.

FIG. 11 shows an isometric view of a patient reference sensor of the second embodiment of the patient reference assembly according to an embodiment of the invention.

FIG. 12 shows a cross-sectional view of the second embodiment of the patient reference assembly according to an embodiment of the invention.

FIG. 13 shows an isometric view of the second embodiment of the patient reference assembly according to an embodiment of the invention.

FIG. 14 shows a cross-sectional view of a third embodiment of a patient reference assembly according to an embodiment of the invention.

FIG. 15 shows an isometric view of a mounting platform of the third embodiment of the patient reference assembly shown in FIG. 14 according to an embodiment of the invention.

FIG. 16 shows a top view of the mounting platform shown in FIG. 15 according to an embodiment of the invention.

FIG. 17 shows a side view of a patient reference sensor of the third embodiment of the patient reference assembly shown in FIG. 14 according to an embodiment of the invention.

FIG. 18 shows an isometric view of the patient reference sensor shown in FIG. 17 according to an embodiment of the invention.

FIG. 19 shows a bottom view of the patient reference sensor shown in FIGS. 17-18 according to an embodiment of the invention.

FIG. 20 shows an embodiment of a mounting platform of a patient reference assembly including a handle according to an embodiment of the invention.

FIG. 21 shows an embodiment of a mounting platform of a patient reference assembly including a wheel-shaped handle according to an embodiment of the invention.

FIG. 22 shows an embodiment of a mounting platform of a patient reference assembly including an adhesive surface mount according to an embodiment of the invention.

FIG. 23 shows an embodiment of a mounting platform of a patient reference assembly including a clamp according to an embodiment of the invention.

FIG. 24 shows an isometric view of a fourth embodiment of a patient reference assembly according to an embodiment of the invention.

FIG. 25 shows an isometric view of a mounting platform of the fourth embodiment of the patient reference assembly according to an embodiment of the invention.

FIG. 26 shows a top view of the mounting platform of the fourth embodiment of the patient reference assembly according to an embodiment of the invention.

FIG. 27 shows an isometric view of a patient reference sensor of the fourth embodiment of the patient reference assembly according to an embodiment of the invention.

FIG. 28 shows a bottom view of the patient reference sensor of the fourth embodiment of the patient reference assembly according to an embodiment of the invention.

FIGS. 4-28 are drawn approximately to scale.

### DETAILED DESCRIPTION

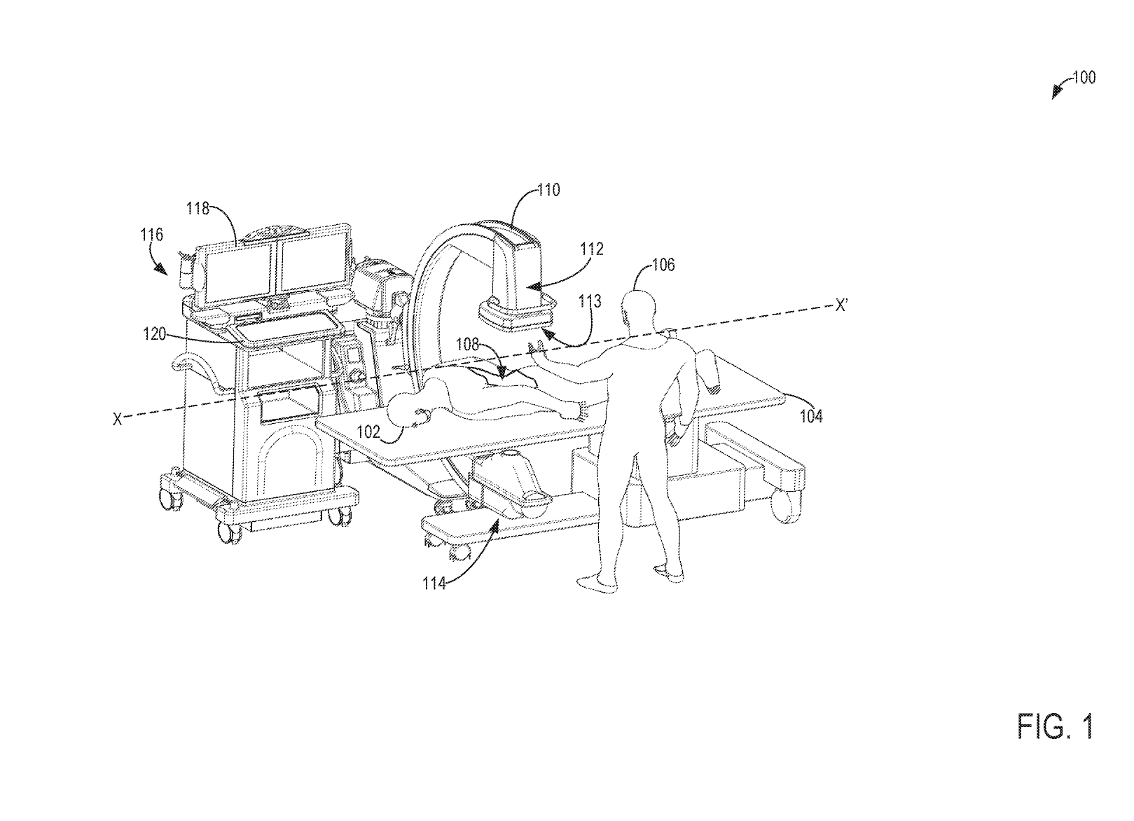

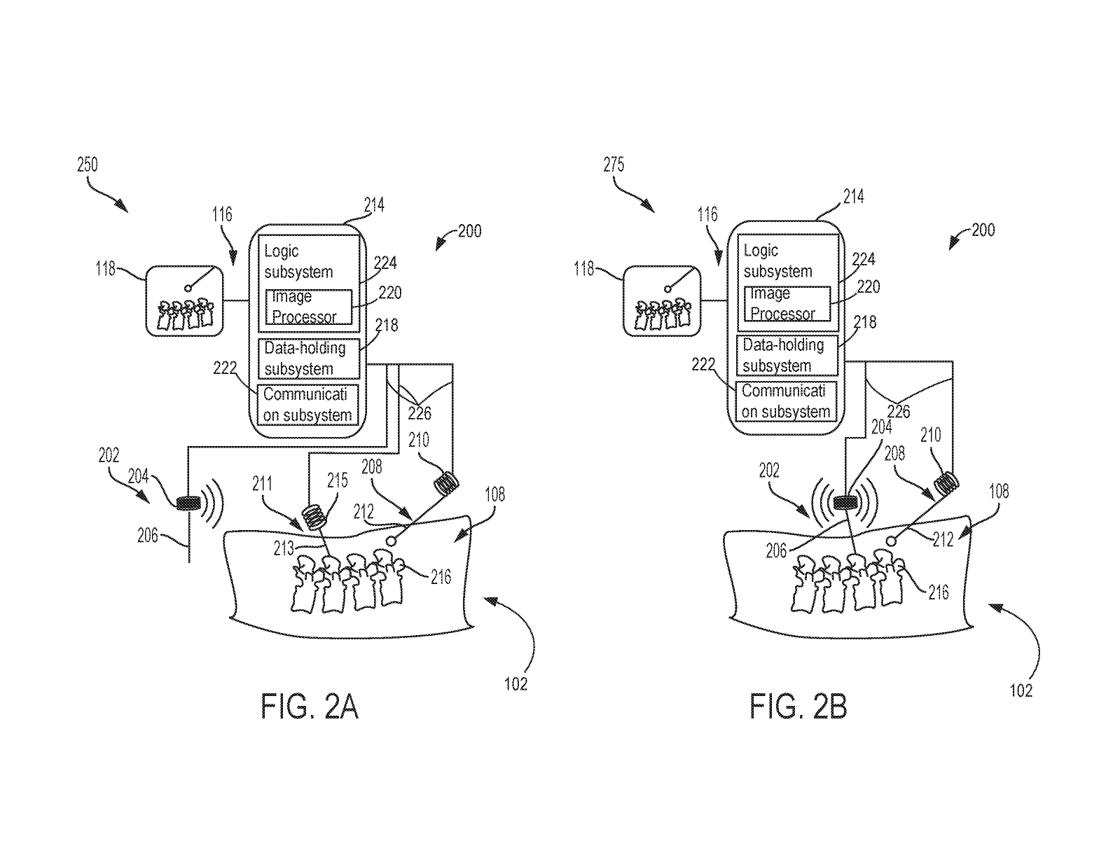

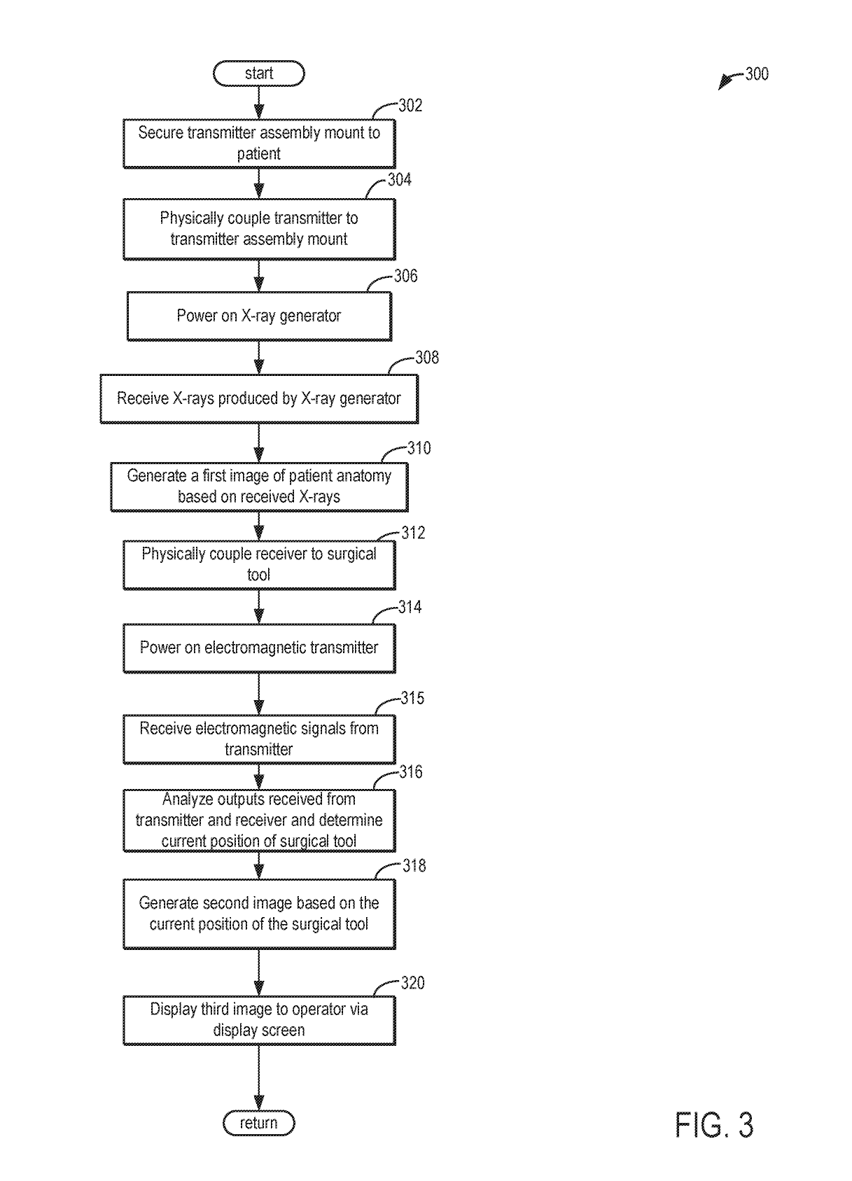

The following description relates to various embodiments of a patient reference assembly for an electromagnetic tracking system for use in image-guided surgery. In some image-guided surgery systems, such as the example system shown in FIG. 1, a C-arm may be used to generate an X-ray image of a patient's anatomy. The electromagnetic tracking system, an example of which is shown in FIGS. 2A-B, may provide an indication of the current location of one or more medical instruments relative to the patient's anatomy. Specifically, estimations of the instruments' current positions may be overlaid onto the X-ray image of the patient's anatomy and displayed to an operator (e.g., surgeon, physician, medical practitioner) as described in the example method shown in FIG. 3. In this way, the operator may continue to adjust the position of the medical instruments within the patient's anatomy based on the displayed images of the instruments, even when the instruments are obscured from sight. Although images of the patient anatomy may be obtained using a C-arm, it should be appreciated that the present techniques may also be useful when applied to images acquired using other imaging modalities, such as tomosynthesis, MRI, CT, and so forth. The present discussion of a C-arm imaging modality is provided merely as an example of one suitable imaging modality.

The electromagnetic tracking system may include a patient reference assembly that functions as both a transmitter sensing and sensed by a receiver in the tracking system, or a receiver sensing and sensed by a transmitter in the tracking system, and as a reference system. FIGS. 4-23 show embodiments of a patient reference assembly for an electromagnetic surgical navigation system (e.g., such as patient reference assembly 202 and electromagnetic tracking system 200 shown in FIGS. 2A-B). As described above, the patient reference assembly may include a patient reference sensor (e.g., which may include one or more electromagnetic coils) and a mounting platform (e.g., mount), where the patient reference sensor and mounting platform include complementary mating interfaces shaped to be removably couple to one another. As a result, the patient reference sensor may be removably coupled with the mounting platform. Additionally, the mounting platform may include an attachment interface for directly coupling the mounting platform to an object, such as a patient (e.g., anatomy of a patient). FIGS. 4-23 show different embodiments for the attachment interface, mating interface of the mounting platform, body shape of the mounting platform, and mating interface of the patient reference sensor. It should be noted that components of the patient reference assembly described herein with regard to one embodiment may be interchanged with patient reference assembly components of an alternate embodiments. For example, a mating interface of a first mounting platform may be used with an attachment interface of a different, second mounting platform. Further, while different embodiments of an exterior housing (e.g., body) structure of the patient reference sensor may be described below, it should be noted that the internal components and functioning of the patient reference sensor (e.g., electronics and electromagnetic coils) may remain the same between embodiments. Further, though similar components may be numbered differently with respect to the different embodiments, similarly named components may have similar functions, for example as described above with reference to FIGS. 1-3. It should also be noted that the patient reference assembly described herein may be implemented as either a transmitter or receiver in the navigation system.

Beginning with FIG. 1, it shows a schematic of an example image-guided surgery system 100. The image guided surgery system 100 includes an electromagnetic tracking system (e.g., such as electromagnetic tracking system 200 described below with reference to FIG. 2) for aiding an operator 106 in performing surgery on a patient 102. The operator 106 may include one or more of a surgeon, physician, surgeon assistant, anesthesiologist, nurse, etc. Patient 102 may lie on table 104 positioned between an X-ray generator 114 and image intensifier or detector 112 of a C-arm 110. The C-arm 110 therefore comprises the generator 114 and detector 112, and generates an image of anatomy 108 of patient 102 based on outputs from the detector 112. The anatomy 108 may include a portion of the patient 102 which is one or more of undergoing surgery, exposed, to be operated on, etc., and as such anatomy 108 may also be referred to in the description herein as operating area 108. Patient 102 may be positioned on table 104, such that anatomy 108 is in-between the generator 114 and detector 112. In this way, when generator 114 is powered on, X-rays pass through anatomy 108 of patient 102.

The C-arm 110 may rotate about axis X-X′ when X-ray generator 114 is energized and emitting X-rays, to generate images of the patient anatomy 108 from multiple angles. Axis X-X′ may be approximately parallel to table 104. Thus, the C-arm 110 rotates around the table 104 and patient 102. An image of the patient anatomy 108 may be obtained based on outputs from the detector 112 during a portion or all of the rotational movement of the C-arm 110 while the generator 114 is powered on. That is, the C-arm may be rotated a threshold number of degrees to obtain an image of the patient anatomy 108 as described in greater detail below with reference to FIG. 3. Specifically, the C-arm 110 may generate a plurality of outputs during the rotating of the C-arm 110, and a three-dimensional image of the patient anatomy 108 may be obtained by compiling the plurality of outputs into a single image. In some examples, the C-arm 110 may be rotated approximately 180 degrees when obtaining an image of the patient anatomy 108. However, in other examples the C-arm 110 may be rotated more or less than 180 degrees. In yet further examples, the C-arm may not be rotated and may remain approximately stationary when obtaining an image of the patient anatomy 108. In such examples where the C-arm 110 remains substantially stationary when the generator 114 is powered on, a single projection image (e.g., two-dimensional image) of the patient anatomy 108 may be obtained.

The X-ray generator 114 produces X-ray radiation that may penetrate the patient anatomy 108 and pass on to the detector 112. As the X-rays pass through the patient 102, the intensity of the X-rays may be attenuated to different degrees. These differences in X-ray intensity may be detected and/or amplified by the detector 112 across a surface 113 of the detector 112. Thus, an image of the patient anatomy 108 may be generated based on the relative intensities of received X-rays distributed across the surface 113 of the detector 112. A computing system 116 may receive and/or process the image data corresponding to the patient anatomy 108 from the detector 112, and may display an image of the anatomy 108 on a display screen 118.

In some examples, the detector 112 may be an analog image intensifier that converts the X-rays received from generator 114 into visible light. In such examples, the surface 113 of the detector 112 may comprise a fluorescent surface which illuminates in response to excitation by X-rays. The brightness or intensity of the surface 113 depends on the intensity of the X-rays striking the surface 113. Thus, as the X-rays generated by the generator 114 strike the surface 113 of the detector 112, the surface 113 may glow or illuminate in proportion to the intensity of the X-rays. Further, the detector 112 may include a camera positioned behind the surface 113. The camera captures a picture of the visible light produced by surface 113 in response to X-ray excitation, and this image is then sent to an image processor and/or storage component of computing system 116.

However, in other examples, the detector 112 may be configured as a flat-panel detector that converts the intensity of received X-rays directly into a digital value. In such examples, the detector 112 does not include a camera, and an image of the patient anatomy 108 may be generated based on the digital signals output from the detector 112. The digital signals output by the detector 112 therefore correspond to the relative intensities of the detected X-rays distributed across surface 113. From the detector 112, the digital signals may be sent to an image processor and/or storage component of the computing system 116. Based on the digital signals received from the detector 112, the computing system 116 may generate an image of the patient anatomy 108 and display the image on display screen 118.

The display screen 118 may be any suitable display screen such as cathode ray tube (CRT), LED, LCD, plasma display, etc. The display screen 118 may positioned such that it faces the operator 106. In this way, the operator 106 can identify and check anatomical details on the images displayed by the display screen 118, such as blood vessels, bones, kidney stones, the position of implants and instruments, etc. Further, the operator 106 may monitor instrument position by watching the display screen 118 during image-guided surgery.

The computing system 116 may include the image processor and various other components such as random access memory (RAM), keep alive memory (KAP), processors, logic subsystems, data-holding subsystems, servers, software instructions, etc., as described in more detail below with reference to FIG. 2. Further, the computing system 116 may include one or more user input devices 120 such as keyboards, mice, buttons, touch screen displays, etc. As described in greater detail below with reference to FIGS. 2 and 3, the computing system 116 may construct a three-dimensional image of the patient anatomy 108 based on outputs from the C-arm 110 obtained during rotation of the C-arm 110, when the generator 114 is powered on. Alternatively, the computing system 116 may construct single projection images of the patient anatomy 108 in examples where the C-arm remains substantially stationary with respect to the patient anatomy 108 when the generator 114 is powered on. Additionally, the computing system 116 may determine a current position and/or orientation of one or more medical instruments based on outputs from an electromagnetic receiver sensor and electromagnetic transmitter of the electromagnetic tracking system. The computing system 116 may overlay an image of the current positions and/or orientations of the one or more medical instruments onto the X-ray image or three-dimensional tomographic image of the patient anatomy 108. This composite image of the patient anatomy 108 and medical instrument position may then be displayed on the display screen 118 to the operator 106.

Turning now to FIGS. 2A and 2B, they show schematics of examples of an electromagnetic tracking system 200 that may be used in an image guided surgery system, such as the image guided surgery system 100 described above with reference to FIG. 1. As such, components of the image guided surgery system 100 described above in FIG. 1 and numbered similarly in FIGS. 2A and 2B may be not be reintroduced or described again in the description of FIGS. 2A and 2B herein. FIGS. 2A and 2B depict example surgical conditions of patient anatomy 108, where tracking system 200 is included to monitor the positions of one or more medical instruments. As depicted in FIGS. 2A and 2B bone 216 of the anatomy 108 is exposed and may be used to physically secure components of the electromagnetic tracking system 200.

The electromagnetic tracking system 200 includes an electromagnetic transmitter and one or more electromagnetic receivers. Specifically, the transmitter may generate a magnetic field when current is provided. The magnetic field produced by the transmitter may induce current to flow in the one or more receivers. Based on the mutual inductances of the transmitter and receiver, a position of the one or more receivers relative to the transmitter may be estimated. Electromagnetic coils that may serve as either the transmitter or the receiver may be included within and/or coupled to each of a medical instrument and a patient reference sensor. Outputs from the electromagnetic coils (e.g., changes in current and/or voltage within the coils) may therefore provide an indication of the position and/or orientation of the instrument relative to the patient anatomy.

FIG. 2A shows a first schematic 250 of an example of the tracking system 200 where an electromagnetic patient reference assembly 202 is positioned proximate and external to the patient anatomy 108. In such examples, a reference coil assembly or reference receiver sensor 211 may be included in the tracking system 200 to increase the accuracy of the system 200. However, FIG. 2B shows a second schematic 275, depicting an alternate example of the tracking system 200, where the patient reference assembly 202 is positioned within and/or coupled to the patient anatomy 108. In such examples, the additional reference receiver sensor may not be included in the tracking system 200. Thus, in some examples, where the patient reference assembly 202 is coupled to the patient anatomy 108, the patient reference assembly 202 may serve as both the electromagnetic transmitter and the reference sensor.

The patient reference assembly 202 may comprise a patient reference sensor 204 and a mount (also referred to herein as a mounting platform) 206 that physically secures the patient reference assembly 202 (e.g., to the anatomy 108 in the example shown in FIG. 2B). Thus, the mount 206 may secure the patient reference assembly 202 so that the patient reference assembly 202 is substantially stationary. Said another way, the mount 206 may physically couple the patient reference assembly 202 to restrict and/or prevent movement of the patient reference assembly 202.

In the example shown in FIG. 2A, the mount 206 may be physically coupled to the patient 102 at a location external to the patient anatomy 108. However, in other examples, the mount 206 may be physically coupled to a location external to the patient 102. For example, the mount 206 may not be coupled to the patient 102 and may instead be coupled to a stationary object external to the patient 102 (e.g., bedside table).

Mount 206 may comprise an attachment interface for securing the mount to the external location such as a pin, clamp, screw, adhesive plate, etc. The patient reference sensor 204 may in some examples be selectively coupled to, and decoupled from, the mount 206, as described below with reference to FIGS. 4-23. That is, the patient reference sensor 204 may be removably coupled to the mount 206. However, in other examples, the patient reference sensor 204 may be permanently secured to the mount 206. In such examples, the patient reference sensor 204 and mount 206 may be integrally formed as a single component.

Similarly, an instrument tracking assembly 208 may include a medical instrument 212 and a tracking sensor 210. The tracking sensor 210 may in some examples be selectively coupled and decoupled from the medical instrument 212. That is, the tracking sensor 210 may be removably coupled to the medical instrument 212. The medical instrument 212 may include one or more of forceps, clamps, retractors, distractors, scalpels, lancets, dilators, suction tips and tubes, injection needles, drills, endoscopes, tactile probes, screw inserters, awls, taps, rod inserters, pedicle probes, etc. However, in other examples, the tracking sensor 210 may be permanently secured to the medical instrument 212. In such examples, the tracking sensor 210 and instrument 212 may be integrally formed as a single component.

Further, the reference receiver sensor 211 may include an electromagnetic receiver 215 and a mount 213. The receiver 215 may in some examples be selectively coupled to and decoupled from the mount 213. That is, the receiver 215 may be removably coupled to the mount 213. However, in other examples, the receiver 215 may be permanently secured to the mount 213. In such examples, the receiver 215 and mount 213 may in some examples be integrally formed as a single component. The receiver 215 may in some examples be the same and/or similar to the tracking sensor 210. However, in other examples, the receiver 215 may be different than the tracking sensor 210. In yet further examples, the receiver 215 may be the same and/or similar to the reference sensor 204.

The reference receiver sensor 211 may be physically coupled to the patient anatomy 108 via the mount 213. Mount 213 may comprise one or more of a pin, clamp, screw, adhesive plate, etc. In some examples, the mount 213 may be physically secured to bone 216 of the patient anatomy 108. However, in other examples, the mount 213 and reference receiver sensor 211 may be physically secured to another portion of the anatomy 108 such as skin, organs, muscle, fat, etc.

Patient reference sensor 204 may comprise any suitable electromagnetic coil arrangement for generating and/or sensing an electromagnetic field. In some examples, the patient reference sensor 204 may be configured as an electromagnetic transmitter. Thus, coils included in the patient reference sensor 204 may generate electromagnetic waves when current flows there-through. In some examples, current may be provided by the computing system 116 via one or more electrical cables. However, in other examples, the patient reference sensor 204 may receive electrical power from another source such as a wall socket, battery, generator, etc. In still further examples, the patient reference sensor 204 may include its own power source, such as a battery. The current may be one or more of DC or AC current. In some examples, the patient reference sensor 204 may be directly electrically coupled to the computing system 116 via one or more electrical cables 226, as shown in FIGS. 2A and 2B. However, in other examples, the patient reference sensor 204 may be wirelessly connected to the computing system 116 (e.g., via Bluetooth, Wifi, etc.).

One or more of the electrical power, current, and voltage supplied to the patient reference sensor 204 may be adjusted to regulate one or more of the intensity, frequency, and wavelength of electromagnetic waves generated by the patient reference sensor 204. In a preferred embodiment the electromagnetic waves generated by the patient reference sensor 204 may be radio waves. However, the frequency of the electromagnetic waves may be altered as desired by adjusting the current supplied to the patient reference sensor 204. Further, the computing system 116 may monitor one or more of the current, voltage, and power of the transmitter via 204, via the direct electrical connection.

The electromagnetic waves generated by the patient reference sensor 204 may be detected by one or more of the tracking sensor 210 of tracking assembly 208 and the receiver 215 of reference receiver sensor 211. Specifically, the electromagnetic field generated by the patient reference sensor 204 may induce current to flow in the tracking sensor 210 and/or receiver 215. The induced current flow in one or more of the tracking sensor 210 and/or receiver 215 may in turn generate electromagnetic fields that induce a change in the current flow in the patient reference sensor 204 (mutual inductance). Thus, the mutual inductances of one or more of the tracking sensor 210, receiver 215, and patient reference sensor 204 may cause changes in current flow therein, and therefore changes in outputs from one or more of the tracking sensor 210, receiver 215, and patient reference sensor 204.

The induced electrical outputs of one or more of the tracking sensor 210 and, receiver 215, and reference sensor 204 may then be used to determine a position of the tracking sensor 210 relative to the patient reference sensor 204. In some examples, outputs from both the patient reference sensor 204 and tracking sensor 210 may be used to determine a position and/or orientation of the tracking sensor 210 relative to the patient reference sensor 204. However in yet further examples, outputs from all of the patient reference sensor 204, receiver 215, and tracking sensor 210 may be used to determine a position and/or orientation of the tracking sensor 210 relative to the patient reference sensor 204. The receiver 215 may therefore serve as a patient reference sensor, providing a reference point, from which the position of the tracking sensor 210 may more accurately be estimated.

However, in other examples, the patient reference sensor 204 may be configured as an electromagnetic receiver, and the tracking sensor 210 may be configured as the electromagnetic transmitter. In such examples, the tracking sensor 210 may be supplied an initial current to generate the electromagnetic field, that may in turn be detected by the reference sensor 204. In yet further examples, the receiver sensor 211 may be configured as the electromagnetic transmitter. Thus, one of the reference sensor 204, tracking sensor 210, or receiver sensor 211 may be configured as an electromagnetic transmitter that generates an electromagnetic field when energized with an electric current.

The patient reference sensor 204 may include three coils arranged in an industry-standard coil arrangement (ISCA). Specifically, the patient reference sensor 204 may contain three approximately co-located, orthogonal quasi-dipole coils. However, in other examples more or less than three coils may be included in the patient reference sensor 204. Further, the orientation and/or arrangement of the coils included within the patient reference sensor 204 may be altered as desired. In some examples, the coils of patient reference sensor 204 may be concentrically positioned relative to one another. Further, the coils may be spaced approximately equally from one another about a center point.

Similar to the patient reference sensor 204, the tracking sensor 210 and receiver sensor 215 may each include three primary coils. Specifically, the tracking sensor 210 and receiver sensor 215 may each contain three approximately co-located, orthogonal quasi-dipole coils. However, in other examples more or less than three primary coils may be included in each of the tracking sensor 210 and receiver sensor 215. The primary coils in each of the tracking sensor 210 and receiver sensor 215 may be aligned substantially perpendicular to each other and may thus define a three-dimensional coordinate system. However, the orientation and/or arrangement of the primary coils included within the receivers 215 may be altered as desired. In some examples, the coils of tracking sensor 210 and receiver sensor 215 may be concentrically positioned relative to one another. Further, the coils may be spaced approximately equally from one another about a center point.

The mutual inductances between each of the coils in the tracking sensor 210 and receiver sensor 215, and each of the coils in the patient reference sensor 204 may be measured and/or estimated by the computing system 116. The position and orientation of the patient reference sensor 204 with respect to the tracking sensor 210 may then be calculated from the resulting mutual inductances of each of those coils and the knowledge of the coil characteristics. In this way, when the tracking sensor 210 is coupled to the medical instrument 212, the position of the medical instrument 212 may be estimated by the computing system 116 based on outputs from one or more of the tracking sensor 210 and receiver sensor 215 and/or patient reference sensor 204.

Computing system 116, may include the display screen 118, and a computing device 214. The computing device 214 includes various hardware and software components for executing instructions and control operations, such as those described below with reference to FIG. 3. For example, the computing device 214 may include a logic subsystem 224, data-holding subsystem 218, image processing subsystem 220, and communication subsystem 222.

Logic subsystem 224 may include one or more processors that are configured to execute software instructions. For example, the logic subsystem 224 may include an image processor 220 for generating images of patient anatomy 108 and/or current positions of the medical instrument 212 based on instructions stored in data-holding subsystem 218 and outputs received from one or more of an X-ray detector (e.g., detector 112 shown in FIG. 1), patient reference sensor 204, and one or more of the tracking sensor 210 and receiver sensor 215. Specifically, the image processor 220 may construct an image of the patient anatomy 108 based on outputs received from the X-ray detector. The image processor 220 may then construct images showing the relative positioning of the medical instrument 212 with respect to the patient anatomy 108 based on outputs from one or more of the tracking sensor 210 and/or re215 and patient reference sensor 204.

Additionally or alternatively, the logic subsystem 224 may include one or more hardware or firmware logic machines configured to execute hardware or firmware instructions. Processors of the logic subsystem 224 may be single or multi-core, and the programs executed thereon may be configured for parallel or distributed processing. The logic subsystem 224 may optionally include individual components that are distributed throughout two or more devices, which may be remotely located and/or configured for coordinated processing.

Data-holding subsystem 218 may include one or more physical, non-transitory devices configured to hold data and/or instructions executable by the logic subsystem 224 to implement the herein described methods and processes. Thus, the methods and routines described below with reference to FIG. 3 may be stored in non-transitory memory of data-holding subsystem 218. When such methods and processes are implemented, the state of data-holding subsystem 218 may be transformed (for example, to hold different data). Further, information such as look-up tables that may be used to implement the herein described methods and processes may be stored in non-transitory memory of the data-holding subsystem 218. For example, product information such as the size, weight, dimensions, length, volume, specifications, distance to tool tip, manufacturer etc., for each type of medical instrument 212 may be stored in non-transitory memory of the data-holding subsystem 218.

Data-holding subsystem 218 may include removable media and/or built-in devices. Data-holding subsystem 218 may include optical memory (for example, CD, DVD, HD-DVD, Blu-Ray Disc, etc.), and/or magnetic memory devices (for example, hard drive disk, floppy disk drive, tape drive, MRAM, etc.), and the like.

It is to be appreciated that data-holding subsystem 218 includes one or more physical, non-transitory devices. In contrast, in some embodiments aspects of the instructions described herein may be propagated in a transitory fashion by a pure signal (for example, an electromagnetic signal) that is not held by a physical device for at least a finite duration. Furthermore, data and/or other forms of information pertaining to the present disclosure may be propagated by a pure signal.

When included, communication subsystem 222 may be configured to communicatively couple the computing system 116 with one or more other computing devices. For example, the communication subsystem 222 may be configured to connect the computing device 214 with one or more of the tracking sensor 210 and receiver sensor 215 and/or patient reference sensor 204. Communication subsystem 222 may include wired and/or wireless communication devices compatible with one or more different communication protocols. Thus, the communication subsystem 222 may communicatively couple the computing system 116 with one or more of the tracking sensor 210 and receiver sensor 215, patient reference sensor 204, and an X-ray detector (e.g., detector 112 shown in FIG. 1) via a wireless or wired connection. As non-limiting examples, communication subsystem 206 may be configured for communication via a wireless telephone network, a wireless local area network, a wired local area network, a wireless wide area network, a wired wide area network, etc.

Turning now to FIG. 2B, it shows an alternate example of the tracking system 200 where the patient reference assembly 202 is physically coupled to the patient anatomy 108 and reference coil assembly 211, described above in FIG. 2A, is not included. Thus, the reference coil assembly 211 may be eliminated from the tracking system 200 in the example shown in FIG. 2B. As such, the patient reference assembly 202 may serve as the patient reference sensor in the example of the tracking system 200 shown in FIG. 2B.

In the example shown in FIG. 2B, mount 206 of patient reference assembly 202 may be physically coupled to bone 216 of the anatomy 108. However, in other examples, the mount 206 may be coupled to other components of the anatomy 108, such as tissue, muscle, blood vessels, fat, organs, etc.

Other than the positioning of the patient reference assembly 202 relative to the patient anatomy 108 and exclusion of the reference coil assembly 211 however, FIG. 2A may be the same and/or identical to FIG. 2A. Thus, the only difference between FIGS. 2A and 2B may be the location at which the patient reference assembly 202 is coupled. As such, components of the tracking system 200 already introduced in the description of FIG. 2A may not be reintroduced or described again in the description of FIG. 2B herein.

In the example of FIG. 2B, the reference coil assembly 211 described above with reference to FIG. 2A is not included in the tracking system 200. As such, the current position of the medical instrument 212 may be based on outputs from one or more of the patient reference sensor 204 and tracking sensor 210 only. Thus, in examples where the patient reference assembly 202 is coupled to the patient anatomy 108, the computing system 116 may estimate the current position of the medical instrument 212 based on outputs received from the tracking sensor 210 and/or patient reference sensor 204.

More specifically, the mutual inductances between each of the coils in the tracking sensor 210, and each of the coils in the patient reference sensor 204 may be measured and/or estimated by the computing system 116. The position and orientation of the transmitter of the tracking sensor 210 with respect to the patient reference sensor 204 may then be calculated from the resulting mutual inductances of each of those coils and the knowledge of the coil characteristics.

For example, when three coils are included in each of the patient reference sensor 204 and tracking sensor 210, the position and orientation of the transmitter tracking sensor 210 may be estimated based on nine resulting mutual inductances. However, in other examples, when more or less than three coils are used in the patient reference sensor 204 and tracking sensor 210, more or less than nine mutual inductances may occur.

Additionally, in examples where the patient reference assembly 202 is coupled to the patient anatomy 108, a secondary coil may be included in the tracking sensor 210 and/or reference sensor 204. The secondary coil oriented such that it is substantially non-parallel with each of the other coils of the sensor 210 or 204 in which it is included. Said another way, the secondary coil may be positioned such that its magnetic axis is not parallel with the magnetic axes of the other receiver coils of the sensor 210 or 204 in which it is included. The fourth coil may be used to resolve hemispherical ambiguity that can occur when using three-coil assemblies in the tracking sensor 210 and patient reference sensor 204. In this way, the computing system 116 may determine a current position of the tracking sensor 210 based on outputs received from one or more of the tracking sensor 210 and patient reference sensor 204.

In some examples, such as when the reference sensor 204 is configured as an electromagnetic transmitter and the tracking sensor 210 is configured as an electromagnetic receiver, the fourth coil may be included in the tracking sensor 210. In other examples, such as when the reference sensor 204 is configured as an electromagnetic receiver and the tracking sensor 210 is configured as an electromagnetic transmitter, the reference sensor 204 may include the fourth coil. In yet further examples, the fourth coil may be included in the tracking sensor 210 regardless of the configuration of the sensors 204 and 210 as transmitters or receivers.

Turning now to FIG. 3, it shows a flow chart of an example method 300 for displaying a position of a medical instrument (e.g., medical instrument 212 shown in FIGS. 2A and 2B) with respect to patient anatomy (anatomy 108 shown in FIGS. 1-2B) during image-guided surgery. A C-arm (e.g., C-arm 110 shown in FIG. 1) may be used to capture an image of the patient anatomy. During image-guided surgery the position of the medical instrument may be estimated using an electromagnetic tracking system (e.g., electromagnetic tracking system 200 shown in FIGS. 2A and 2B). Specifically, the position of the medical instrument may be estimated based on outputs from electromagnetic coil assemblies included within each of a tracking sensor (e.g., tracking sensor 210 shown in FIGS. 2A and 2B) and a patient reference sensor (e.g., patient reference sensor 204 shown in FIGS. 2A and 2B). Specifically, a first coil assembly may be included in the reference sensor of a patient reference assembly (e.g., patient reference assembly 202 shown in FIGS. 2A and 2B) and a second coil assembly may be included in the tracking sensor of a tracking assembly (e.g., tracking assembly 208 shown in FIGS. 2A and 2B) of the tracking system.

One of either the tracking sensor or the patient reference sensor may be configured as an electromagnetic transmitter, while the sensor not configured as an electromagnetic transmitter may be configured as an electromagnetic receiver. Thus, in some examples, the tracking sensor may be configured as a receiver and the patient reference sensor may be configured as a transmitter. However, in other examples, the tracking sensor may be configured as a transmitter and the patient reference sensor may be configured as a receiver. An image of the patient anatomy, including the current position of the medical instrument with respect to the anatomy, may then be displayed to a surgeon or other medical personnel based on outputs from the patient reference sensor and tracking sensor.

Portions or all of method 300 may be stored in non-transitory memory (e.g., data-holding subsystem 218 shown in FIGS. 2A and 2B) of a computing device (e.g., computing device 214 shown in FIGS. 2A and 2B). As such, portions or all of method 300 may be executed by the computing device to display a position of the medical instrument relative to the patient anatomy to a medical operator (e.g., operator 106 shown in FIG. 1).

Method 300 begins at 302 which comprises securing a mounting platform (e.g., mounting platform 206 shown in FIGS. 2A and 2B) of the patient reference assembly to a patient (e.g., patient 102 shown in FIGS. 1-2). More specifically, the method 300 at 302 may comprise physically coupling the patient reference assembly mounting platform to an operating area (e.g., anatomy 108 shown in FIGS. 1-2) of the patient. Thus, the method at 302 comprises physically coupling the mounting platform of the patient reference assembly to the patient. The mount may be secured to the patient by one or more of screwing, clamping, and securing (e.g., via an adhesive) the mounting platform to the patient. However, other suitable mechanical/adhesive linkages may be used to secure the mounting platform to the patient. For example, the mounting platform may be threaded and may be secured to bone (e.g., bone 216 shown in FIGS. 2A and 2B) by screwing the mount into the bone. Specifically, the mounting platform may be coupled to the spine and/or Iliac Crest of the patient. However, in other examples, the mounting platform may be secured to another part of the patient, such as skin. In yet further examples, the mounting platform may be secured to a location external to the patient.

After securing the mounting platform of the patient reference assembly to the patient at 302, method 300 may then continue to 304 which comprises physically coupling the patient reference sensor to the patient reference assembly mounting platform. As described in greater detail below with reference to FIGS. 4-23, the patient reference sensor and the mounting platform may include mating mechanical interfaces that physically couple and decouple the two components when an external force is applied. For example, the patient reference sensor and mounting platform may include complementary mating elements (e.g., recessed slots 1328 and mating arms 1338 shown in FIGS. 13-19) that couple and decouple the patient reference assembly mounting platform and patient reference sensor when a force is provided by a surgeon or other medical personnel. Specifically, the patient reference sensor and patient reference assembly mounting platform may be coupled and decoupled from one another by rotating the two components relative to one another, thereby manipulating the mating elements. When the patient reference sensor and mounting platform are physically coupled to one another, the two components are attached and mechanically linked to one another, thus forming the patient reference assembly.

Method 300 may then proceed from 304 to 306 which comprises powering on an X-ray generator (e.g., generator 114 shown in FIG. 1) of the C-arm. In some examples, the method 300 at 306 may comprise holding the C-arm substantially stationary while powering on the X-ray generator. However, in other examples, the method 300 at 306 may comprise swiveling the C-arm a threshold number of degrees while powering on the X-ray generator. Thus, as described above with reference to FIG. 1, in some examples, the X-ray generator may be powered on while rotating the C-arm about a central rotational axis (e.g., axis X-X′ shown in FIG. 1). More specifically, in some examples, the method 300 at 306 may comprise swiveling the C-arm from a first position to a second position and powering on the X-ray generator for the duration of the movement from the first position to the second position. However, in other examples, the C-arm may be powered on for only a portion of the duration of the movement from the first position to the second position. Powering on the C-arm may comprise providing electrical power (e.g., voltage and current) to the C-arm.

In some examples, the threshold number of degrees that the C-arm may be rotated while powering on the X-ray generator may be approximately 180 degrees. However, in other examples, the threshold number of degrees may be greater or less than 180 degrees. In some examples, the threshold number of degrees may be determined based on surgical operating conditions such as a desired image quality, patient size and weight, patient anatomy, type of surgical operation, etc. However, in other examples, the threshold may be a preset number of degrees.

In examples where the C-arm is held substantially stationary while powering on the X-ray generator, the X-ray generator may be powered on for a threshold duration, and then after the duration, the X-ray generator may be turned off. However, in examples where the C-arm is rotated while powering on the X-ray generator, the X-ray generator may be turned off once the C-arm has been rotated the threshold number of degrees or has completed its rotation. That is, electrical power provided to the X-ray generator may be terminated.

While the X-ray generator is powered on at 306, an X-ray detector (e.g., detector 112 shown in FIG. 1) of the C-arm may detect X-rays produced by the X-ray-generator at 308. Thus, the method 300 at 308 may comprise receiving X-rays produced by the X-ray generator. In this way, 306 and 308 may be executed approximately simultaneously. As described above with reference to FIG. 1, the X-ray detector may convert the received X-ray intensity into a digital output.

After the X-ray generator is powered off at 306, and the X-rays are received at 308, method 300 may then proceed to 310 which comprises generating a first image of the patient anatomy based on the received X-rays. Specifically, as described above with reference to FIG. 1, an image of the patient anatomy may be generated based on the relative intensities of X-rays received at the received at the X-ray detector at different points along the detection medium (e.g., surface 113 shown in FIG. 1). In some examples, such as examples where the C-arm is held substantially stationary when powering on the X-ray generator at 306, the first image may be a two dimensional image. However, in other examples, such as examples where the C-arm is rotated when powering on the X-ray generator, the first image may be a three dimensional image that may be compiled based on the X-rays received from different angles and positions during the rotation of the C-arm. In such examples, the method 300 at 310 may comprising compiling outputs received from the detector during the rotation of the C-arm while the X-ray generator was powered on, into a three dimensional image.

Method 300 may then continue from 310 to 312 which comprises physically coupling the tracking sensor to a medical instrument (e.g., medical instrument 212 shown in FIGS. 2A and 2B). Similar to the reference sensor and patient reference assembly mounting platform, the tracking sensor and medical instrument may include mating mechanical interfaces that physically couple and decouple the two components when an external mechanical force is applied. For example, one or more of the tracking sensor and/or medical instrument may include an adjustable snapping latch. The tracking sensor and medical instrument be coupled and decoupled from one another by manipulating the snapping latch.

After 312, the method 300 may continue to 314 which comprises powering on the transmitter and producing electromagnetic signals. Specifically, powering on the transmitter may comprise flowing current through the electromagnetic coils included in the transmitter. Thus, the method 300 at 314 may comprise providing electrical power (e.g., voltage and/or current) to the transmitter. By powering on the transmitter, the transmitter may generate an electromagnetic field and produce electromagnetic radiation or waves. In some examples, the electromagnetic waves may be radio waves.

After powering on the transmitter at 314, the method 300 may then continue to 315 which comprises receiving the electromagnetic signals from the transmitter. As explained above with reference to FIG. 2A and 2B, powering on the transmitter and generating an electromagnetic field may induce current to flow in the electromagnetic coils of the receiver (mutual inductance). Thus, the electromagnetic signals generated by the transmitter may be received by the receiver at 315. Further, the induced current flow in the receiver, may correspondingly cause changes in the electrical current of the coils of the transmitter due to mutual inductance between the transmitter and receiver. Thus, the outputs (e.g., current and/or voltage) from the transmitter and the receiver may be affected and/or changed due to the current supplied to the transmitter. As the position and/or orientation of the transmitter relative to the receiver changes, the outputs from the transmitter and receiver may change.

Method 300 may then continue from 315 to 316 which comprises analyzing the outputs from the transmitter and receiver, and determining the current position of the medical instrument based on the outputs. As explained above, the outputs may be in the form of a voltage and/or electrical current. Thus, the method 300 at 316 may first comprise estimating a position of the receiver relative to the transmitter based on the outputs received from the transmitter and the receiver. However, in other examples, the method at 316 may comprising estimating a position of the transmitter relative to the receiver based on the outputs received from the transmitter and receiver. Then, a current position and/or orientation of the medical instrument may be estimated based on known geometric transformation relating the position of the receiver or transmitter (whichever is coupled to the medical instrument) to a position of the medical instrument. More simply, the outputs received from the transmitter and receiver may be calibrated to determine the current position of the medical instrument. However, it should be appreciated that in other examples, the position of the transmitter or receiver may be estimated based on outputs from either the transmitter or the receiver, and that outputs from both the transmitter and receiver may not be used to estimate the position of one relative to the other.

Based on the current position of the medical instrument determined at 316, method then proceeds to generate a second image at 318. The second image may be an image of the medical instrument overlaid onto the first image. Specifically, based on the known size, and dimensions of the medical instrument, an image of the medical instrument may be constructed based on the current position of the medical instrument estimated at 316, where the current position of the medical instrument may be determined based on the estimated positions of the receiver and/or transmitter relative to one another. Thus, the second image may show the current position of the medical instrument relative to the patient anatomy. In this way, both the patient anatomy, and position of the medical instrument may be estimated.

It is important to note that 314, 316, and 318 may be executed approximately continuously while the transmitter is powered on. Thus, the method 300 may return back to 315 and continue to determine the current position of the medical instrument as long as the transmitter remains powered on. Thus, the position of the medical instrument may be updated based on the most recent outputs received from one or more of the transmitter and receiver to reflect the most recent position of the medical instrument. In this way, estimates of the position of the medical instrument may be updated approximately continuously. However, in other examples, estimates of the position of the medical instrument may be updated at regular time intervals or after a pre-set duration has expired since a most recent estimate.

After generating the second image at 318, method 300 may continue to 320 which comprises displaying the second image to the medical operator via a display screen (e.g., display screen 118 shown in FIGS. 1-2B). Thus, the method 300 at 318 comprises displaying an image of the patient anatomy, with a current or more recent position of the medical instrument. Thus, a visual representation of the patient anatomy and medical instrument position may be presented to the medical operator in either a two dimensional or three dimensional image.

As explained above at 306, the C-arm may be rotated to acquire an image of the patient anatomy from a different vantage point. However, in some examples, the C-arm may be rotated while the transmitter is powered on and the position of the medical instrument is being estimated. Thus, in some examples, 306 may be executed after powering on the transmitter. For example, the method 300 at 320 may comprise rotating the C-arm and powering on the C-arm for a duration to acquire a new image of the patient anatomy. In some examples, multiple X-ray images from different angles may be combined to create three dimensional images of the patient anatomy.

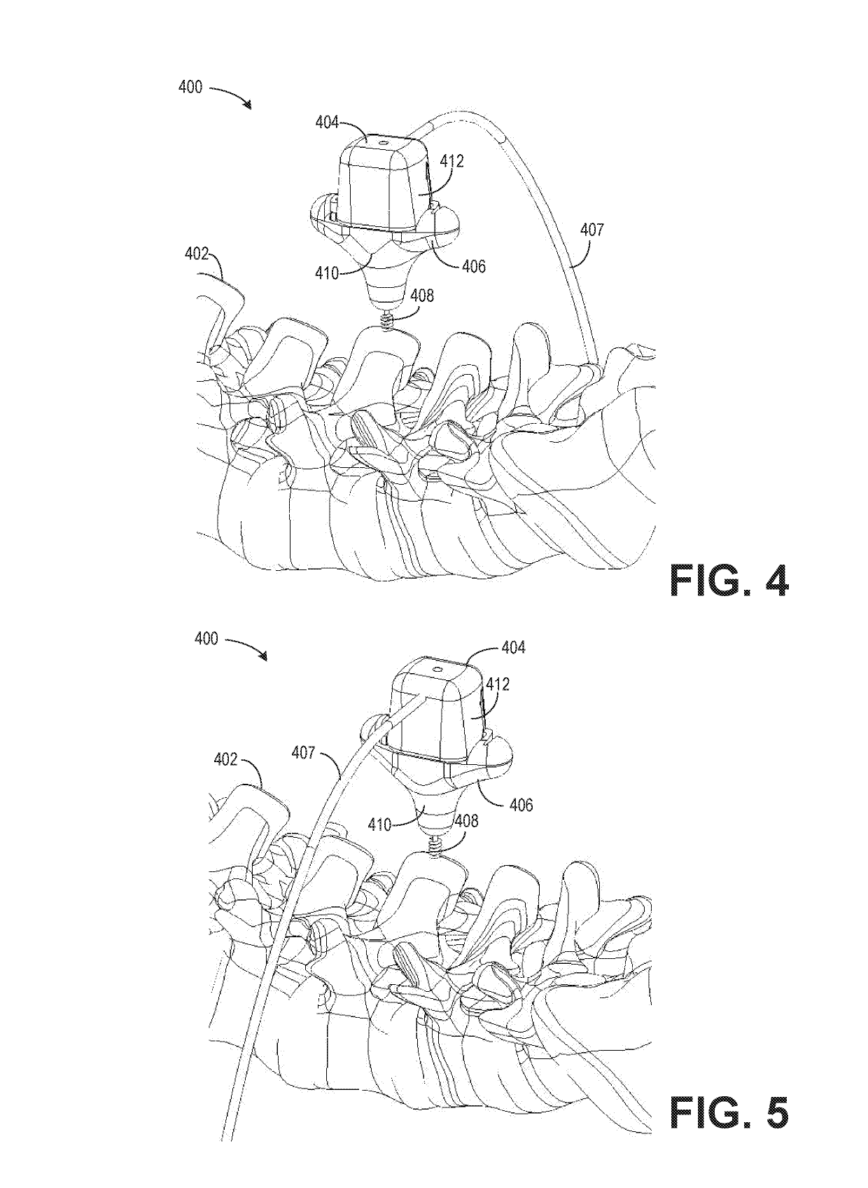

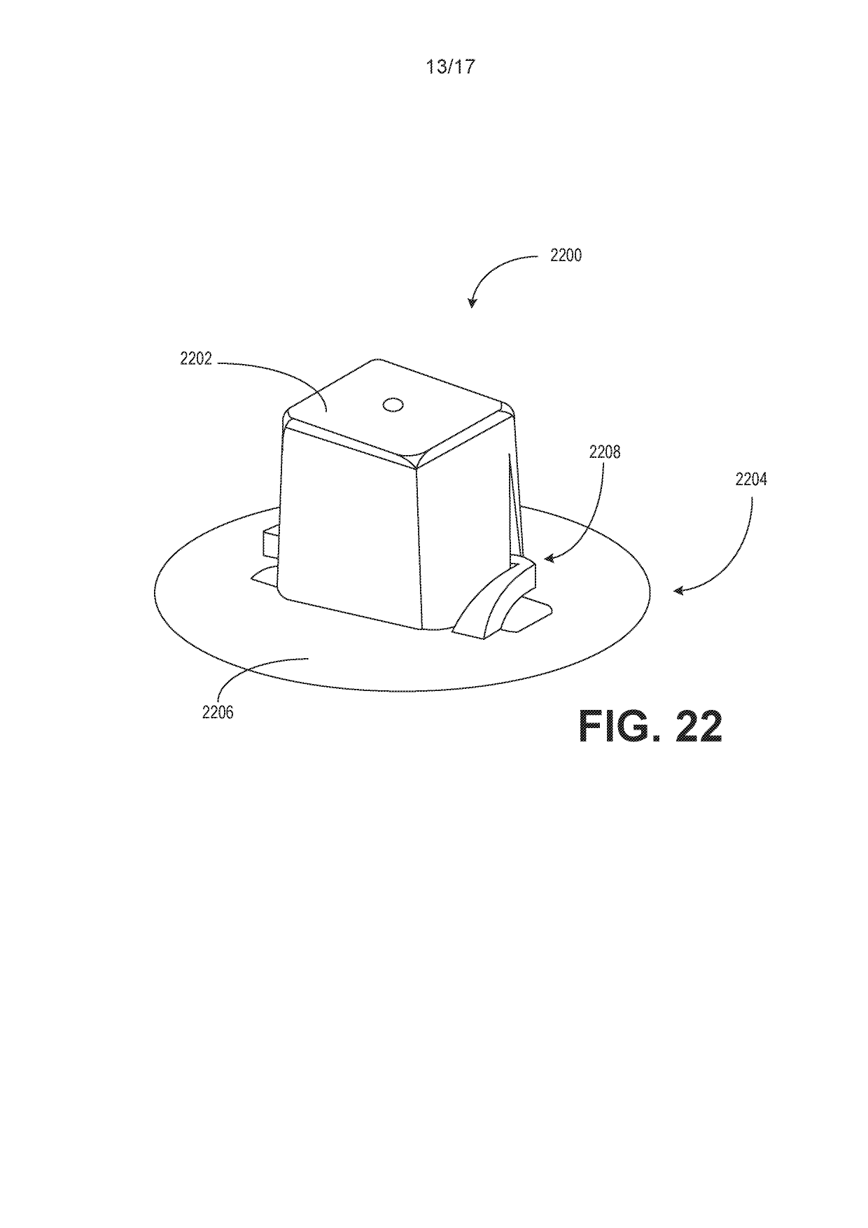

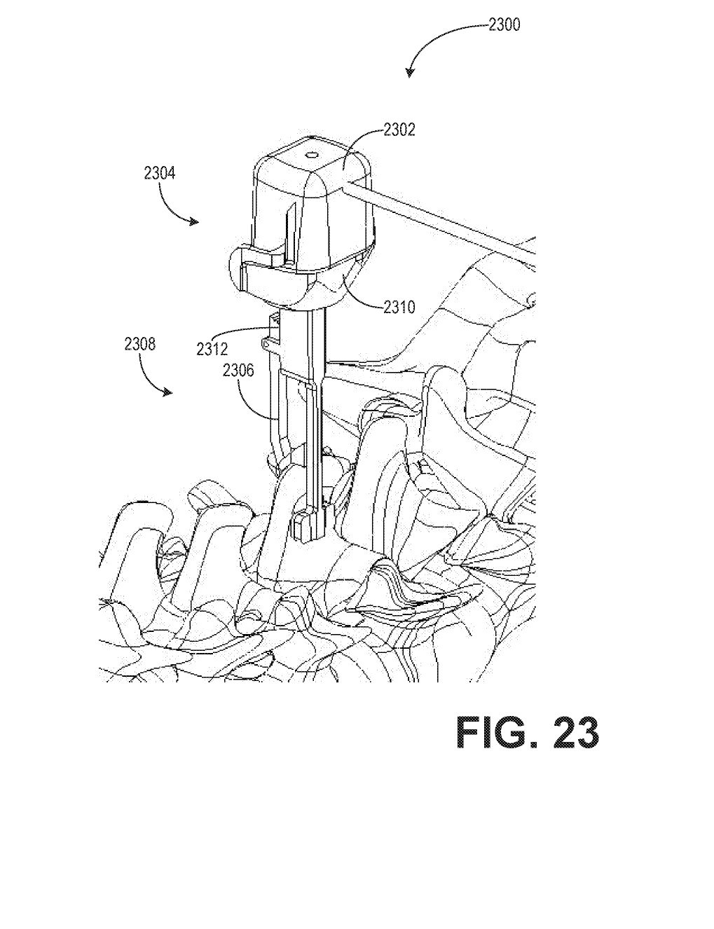

FIGS. 4-5 show an embodiment of a patient reference assembly 400 mounted (e.g., directly coupled) to a spine 402. The patient reference assembly may include a patient reference sensor 404 (e.g., such as transmitter 204 shown in FIG. 2B or receiver 215 shown in FIG. 2A) removably coupled to a mounting platform 406 (e.g., such as mount 206 shown in FIG. 2B). The patient reference sensor includes an electromagnetic coil that serves as one of a transmitter or receiver. Thus, the patient reference assembly may also be referred to herein as a transmitter or receiver assembly that is fixed to the patient via the mounting platform 406. An electrical cable 407 (e.g., such as electrical cable 226 shown in FIG. 2B) is directly coupled to an exterior housing 412 of the patient reference sensor 404 and may couple to a computing system of the electromagnetic surgical navigation system, such as computing system 116 shown in FIGS. 2A-B. The mounting platform 406 includes an attachment interface 408 coupled to a body 410 of the mounting platform 406. The attachment interface 408 is directly coupled to the body 410 at a first end of the attachment interface 408 and directly coupled to the spine 402 at an opposite, second end of the attachment interface 408. As shown in FIGS. 4-5, the attachment interface 408 is a bone screw including a plurality of threads that are adapted to screw into a bone in order to rigidly hold the mounting platform 406 in place relative to the spine 402 of the patient. In alternate embodiments, as shown at FIGS. 22-23 (described further below), the attachment interface 408 may be a different mechanical element capable of interfacing with anatomy of the patient, such as a device that utilizes multiple screw threads for added rigidity, a clamp or an adhesive plate that can be attached to the patient's skin.

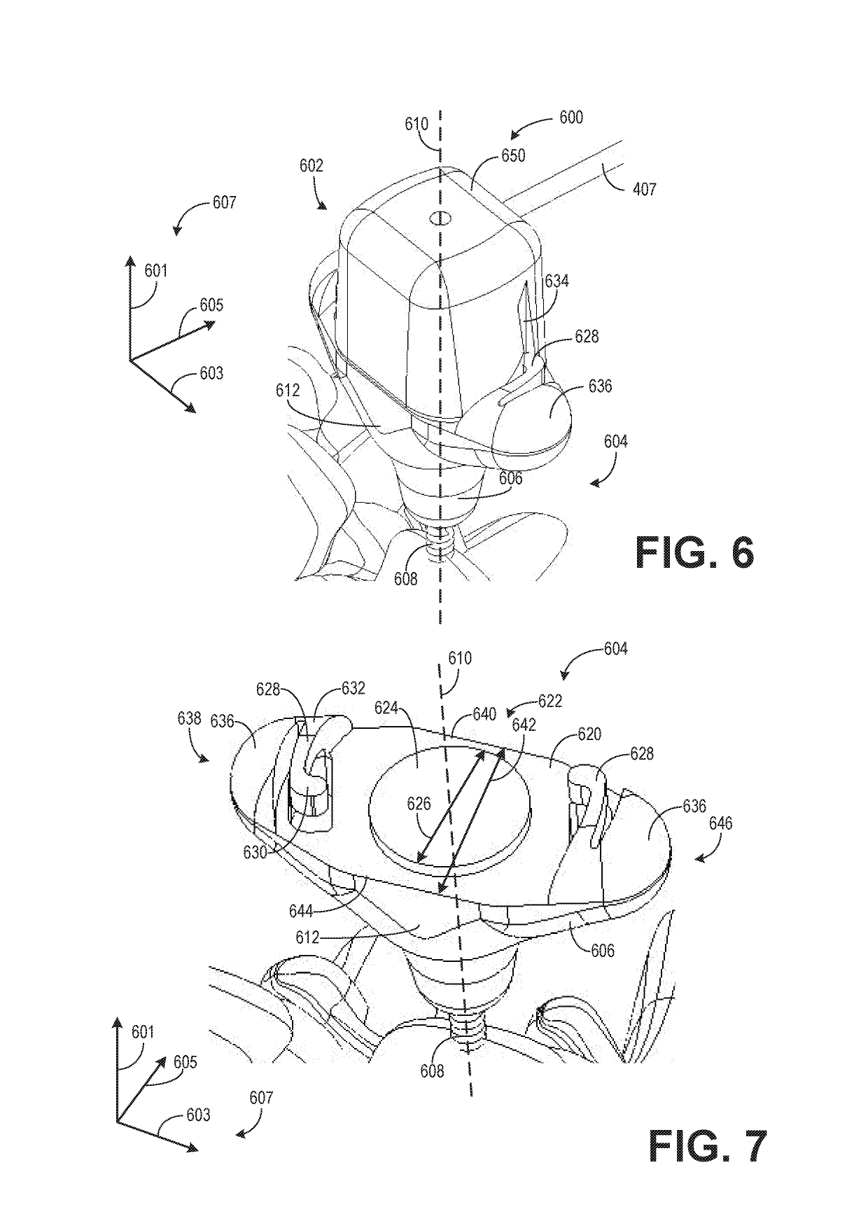

The body 410 of the mounting platform 406 may be shaped to enable a user to attach the mounting platform to the patient's anatomy. For example, in the example shown in FIGS. 4-5, the attachment interface 408 is a screw that must be screwed (e.g., via multiple rotations) into the spine 402. The body 410 is ergonomically shaped to fit within a user's hand and enable to user to attach the mounting platform 406 (e.g., via screwing into the bone, in this example) via holding and turning the body 410 alone, without the aid of additional installation tools. Different embodiments of a shape of the body 410 for attaching the mounting platform 406 to the patient without additional tools are shown in FIGS. 6-7, 13-16, 20, and 21, as explained further below.

Once installed (e.g., coupled to the patient) via the attachment interface 408, the mounting platform 406 may be secured in place and may not move (e.g., re-orient in space) until a user removes the mounting platform 406 from the spine 402. However, the patient reference sensor 404 may be uncoupled from the mounting platform 406 and re-oriented on the mounting platform 406 or moved to a different mounting platform coupled to the patient in a different location than shown in FIGS. 4-5. FIG. 4 shows the patient reference sensor 404 coupled to the mounting platform 406 in a first orientation where the electrical cable 407 extends over a first side of the spine (e.g., into the page). FIG. 5 shows the patient reference sensor 404 coupled to the mounting platform 406 in a different, second orientation where the electrical cable 407 extends over an opposite, second side of the spine (e.g., out of the page). The second orientation of the patient reference sensor 404 is approximately 180 degrees different than the second orientation. In this way, a user may uncouple the patient reference sensor 404 from the mounting assembly 406 and re-couple the patient reference sensor 404 to the mounting sensor 406 in a different orientation in order to change the positioning of the electrical cable 407. This re-orientation may occur during a surgical procedure without having to take the time and energy to re-install the entire patient reference assembly. This may simplify the surgical procedure for the user and enable the user more freedom (e.g., space) to perform necessary operations. As explained further below with reference to FIGS. 6-23, the symmetric nature of the respective mating elements on the mounting platform 406 and patient reference sensor 404 enables the patient reference sensor 404 to be mounted in two different orientations on the same mounting platform 406. For example, the mounting platform 406 may include a first and second mating element, each of which are able to couple to either and each of two corresponding mating elements on the patient reference sensor 404. This concept will be explained in further detail below.

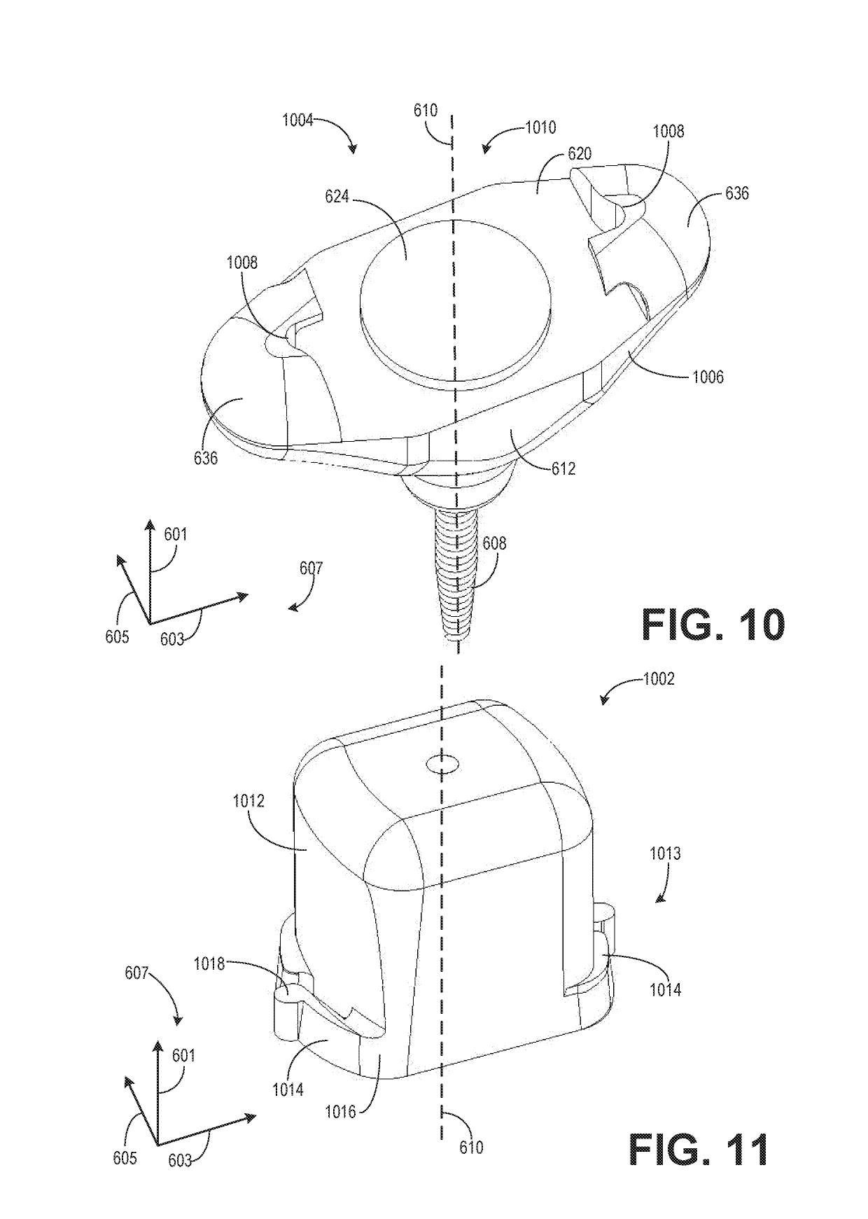

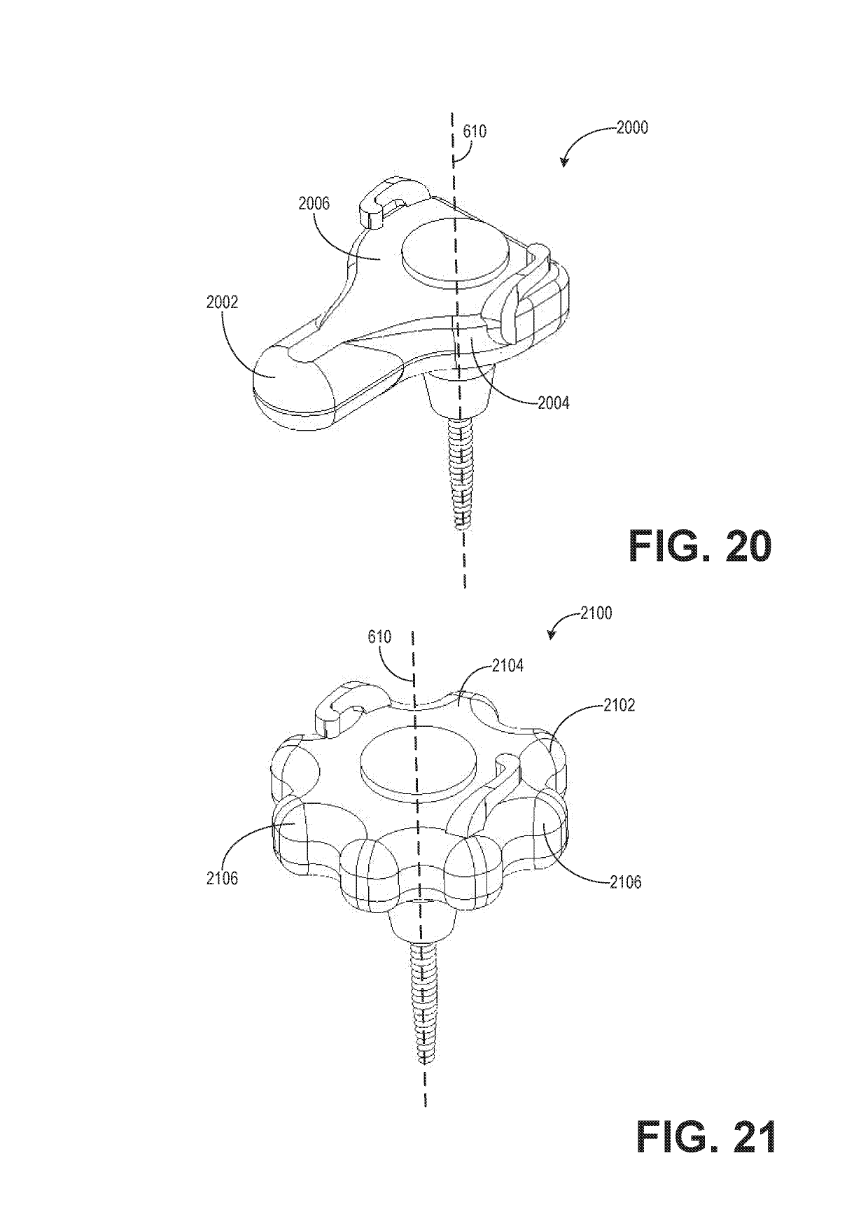

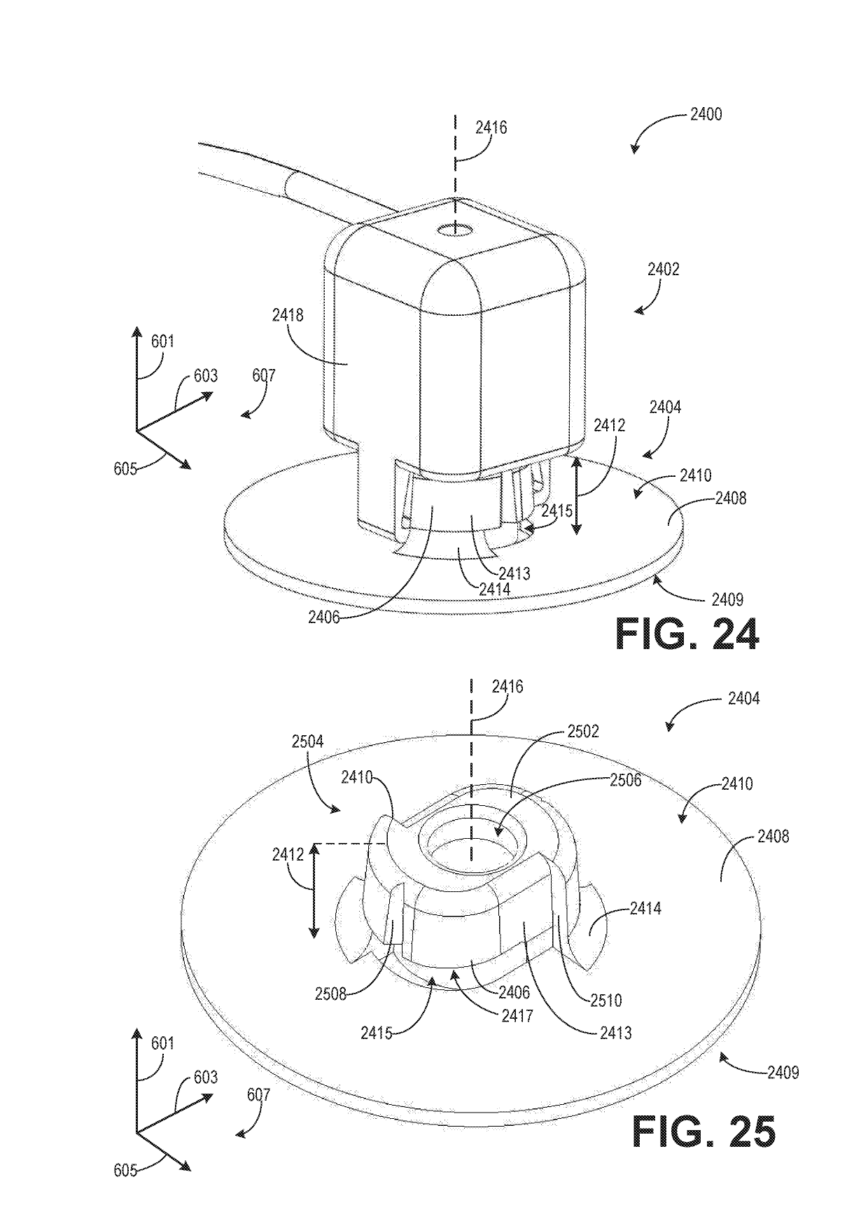

FIGS. 6-9 show a first embodiment of a patient reference assembly, FIGS. 10-12 show a second embodiment of a patient reference assembly, FIGS. 13-19 show a third embodiment of a patient reference assembly, and FIGS. 24-28 show a fourth embodiment of a patient reference assembly. These three different patient reference assembly embodiments may have similar components and corresponding functions as those described above with reference to FIGS. 4-5. The first and second embodiments shown in FIGS. 6-9 and FIGS. 10-12, respectively, have a mounting platform with a similar body structure. The third embodiment shown in FIGS. 13-19 shows a different mounting platform body structure. The fourth embodiment shown in FIGS. 24-28 has a similar reference sensor structure to the third embodiment, but a different mounting platform structure and attachment interface are shown. Additionally, FIGS. 20-21 show an additional two different embodiments of the body structure of the mounting platform. The embodiments of the patient reference assembly shown in FIGS. 6-21 all have the same attachment interface portion of the mounting platform (e.g., a screw). Though, it should be noted that this attachment interface may be replaced with an alternate attachment interface. FIGS. 22-23 show two additional attachment interface embodiments for the patient reference assembly.

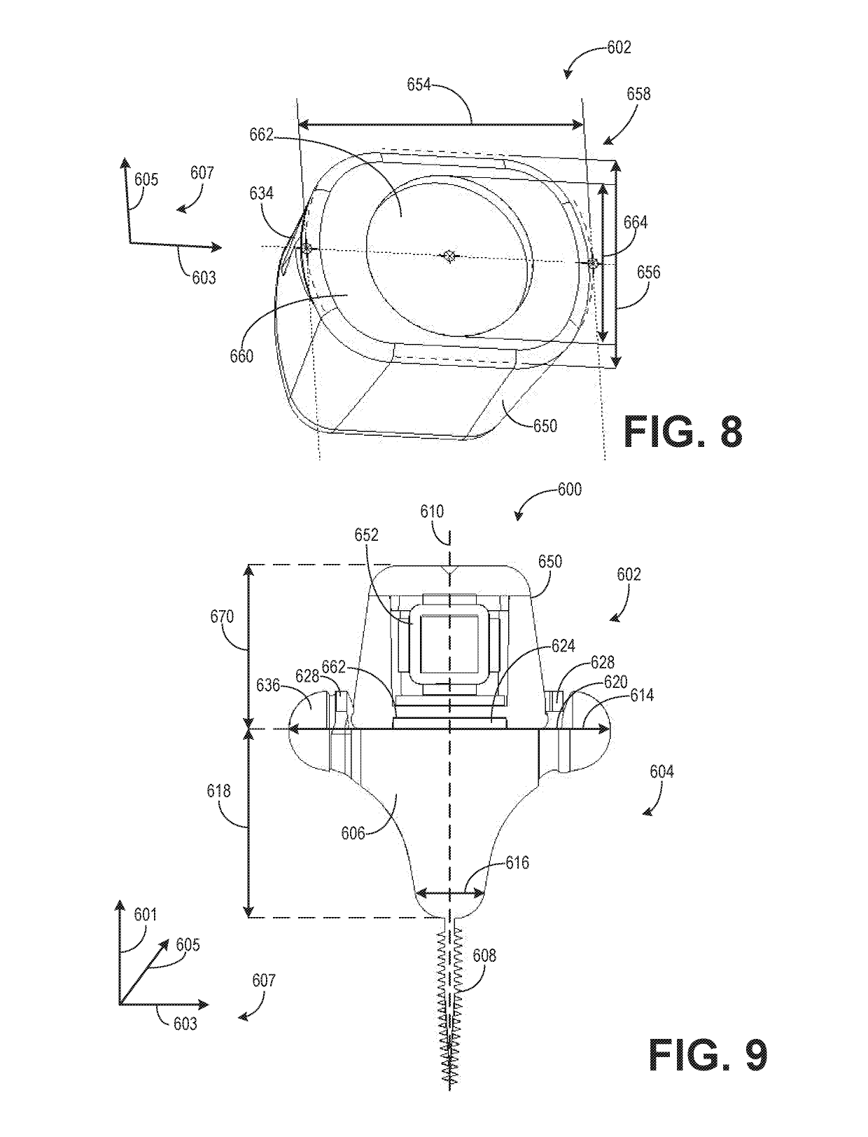

Turning first to FIGS. 6-9, the first embodiment of a patient reference assembly 600 is shown. FIG. 6 shows an isometric view of the patient reference assembly 600 including a patient reference sensor 602 removably coupled to a mounting platform 604. FIG. 7 shows an isometric top view of the mounting platform 604, FIG. 8 shows a bottom view of the patient reference sensor 602, and FIG. 9 shows a cross-sectional view of the patient reference assembly 600. FIGS. 6-9 (as well as many of FIGS. 10-23) show, for reference, an axis system 607 displaying a vertical axis 601, horizontal axis 603, and lateral axis 605. The patient reference assembly 600 also includes a central axis 610, where the central axis 610 is also a central axis of the patient reference sensor 602 and mounting platform 604.

The mounting platform 604 includes a body 606 shaped for attaching (e.g., installing) the mounting platform 604 on the patient's tissue (e.g., anatomy) without the use of a secondary installation tool. Specifically, in the embodiments shown in FIGS. 6-7 and 9, the body 606 is oblong with rounded ends at a top of the mounting platform (relative to the vertical axis 601 and a surface on which a patient to which the mounting platform is coupled sits). The body 606 then tapers inward (toward the central axis 610) in a downward direction (e.g., negative direction along vertical axis 601) along the central axis 610. A bottom portion of the body 606, which is closest to an attachment interface 608 is a narrowest part of the body 606 and has a relatively circular cross-section in a plane of the horizontal axis 603 and lateral axis 605. A middle portion of the body 606 includes a protrusion 612 (as seen in FIGS. 6 and 7) extending outward from the body in a direction of the negative lateral axis 605, relative to the central axis 610. The protrusion 612 is rounded and shaped to fit between fingers of a user when installing the mounting platform 604 on the patient. In an alternate embodiment, the protrusion 612 may extend from the opposite side of the body than shown in FIGS. 6 and 7. In yet another embodiment, both sides (e.g., long sides) of the body 606 may include a protrusion 612 such that there are two protrusions 612.

As seen in FIG. 9, the oblong, top portion of the body 606 has a first length 614 in a direction of the horizontal axis 603 which is the longest part of the patient reference assembly 600. A bottom portion of the body 606 has a second length 616, smaller than the first length 614. The body 606 tapers inward toward the central axis 610 from the first length 614 to the second length 616 along a height 618 of the body 606. As shown in FIGS. 6-7 and 9, the outer surfaces of the body 606 have rounded edges, thereby increasing comfort for a user holding and installing the mounting platform 604.

The attachment interface 608 is coupled to and extends from a bottom of the body 606. As shown in FIGS. 6-7 and 9, the attachment interface 608 is a bone screw; however, in alternate embodiments a difference attachment interface 608 (such as a bone clamp, bone pin, etc.) may be coupled to a bottom of the body 606. In one example, the body 606 may be over-molded on the attachment interface 608. Said another way, the body 606 may be composed of a plastic material which is molded around an end of the attachment interface 608 which may be a metal screw (e.g., composed of titanium or stainless steel). The body 606, including the protrusion 612, may form a handle of the mounting platform 604 for screwing the attachment interface 608 into the patient's bone in order to rigidly fix the mounting platform 604 to the patient without the use of a secondary attachment tool. In this way, only a user's own force may be used to fix the mounting platform 604 to the patient.

The body 606 includes a top surface 620 arranged opposite a bottom surface of the body 606 from which the attachment interface 608 extends. The top surface 620 is oval-shaped and at least a portion of the top surface 620 is planar. The top surface 620 includes a first mating interface 622. The first mating interface 622 includes a central, raised platform (e.g., step) 624. As shown in FIG. 7, the raised platform 624 is circular, centered along the central axis 610, and has a diameter 626. The raised platform 624 extends outward and upward in a direction of the vertical axis 601 from the top surface 620. In alternate embodiments, the top surface 620 may be planar in the center of the top surface 620 and may not include the raised platform 624.

The first mating interface 622 also includes a pair of mating elements 628. The pair of mating elements 628 may also be referred to herein as an interlocking interface of the mounting platform 604. In the embodiment shown in FIGS. 6-7 and 9, the mating elements 628 are hinges (e.g., interlocks) with a curved, hook end 630 and a base end 632 directly coupled to the body 606. The hook ends 630 are free ends which are shaped to mate with complementary mating elements 634 on the patient reference sensor 602, as described further below, when the patient reference sensor 602 is directly and removably coupled with the mounting platform 604. The mating elements 628 are raised above the planar portion of the top surface 620. For example, the base ends 632 are coupled to rounded ends 636 of the body 606 which are raised above the top surface 620. As shown in FIG. 7, the two mating elements 628 are shaped the same and symmetrically positioned on the top surface 620, around the central axis 610. Specifically, each of the two mating elements 628 are spaced apart from one another and arranged on opposite sides of the raised platform 624. For example, a first mating element of the two mating elements 628 is positioned at a first end 638 of the top surface 620 with the base end 632 coupled to a first side 640 of the top surface 620 and the hook end 630 extending across a width 642 (in a direction of the lateral axis 605) to a second side 644 of the top surface 620 (where the second side 644 is opposite the first side 640). A second mating element of the two mating elements 628 is positioned at a second end 646 of the top surface 620 with the base end 632 coupled to the second side 644 of the top surface 620 and the hook end 630 extending across the width 642 (in a direction of the lateral axis 605) to the first side 640 of the top surface 620. In this way, the two mating elements 628 are oppositely arranged and symmetric about the central axis 610.

As shown in FIGS. 6, 8, and 9, the patient reference sensor 602 includes an exterior (e.g., outer) housing 650 encasing and surrounding internal components of the patient reference sensor 602. As described above with reference to FIGS. 2A-B and shown in FIG. 9, the patient reference sensor 602 may include one or more electromagnetic coils 652 within the interior of the patient reference sensor 602 and one or more electrical leads exiting the internal components. A PCB may be used to mount the one or more coils and provide termination for the electrical leads. The electromagnetic coils 652 are wound/formed around either a plastic or ferrite bobbin. An electrical cable 407 extends from within the patient reference sensor 602 and out of the housing 650. However, in alternate embodiments the patient reference sensor 602 may be wireless and not include the electrical cable 407. As shown in FIG. 8, a length 654 of the patient reference sensor 602 is shorter than length 614 of the body 606. Additionally, a width 656 of the patient reference sensor 602 may be approximately the same as the width 642 of the body 606.

As seen in FIGS. 6 and 8, the patient reference sensor 602 includes a second mating interface 658 including a central recess (e.g., depression or cavity) 662 centrally positioned along the central axis 610 and recessed into a bottom surface 660 of the patient reference sensor 602. The central recess 662 includes a diameter 664 that is slightly larger than diameter 626 of the raised platform 624 so that the central recess 662 may receive and fit around (and mate with) the raised platform 624 (as seen in FIG. 9). The second mating interface 658 also includes a pair of mating elements 634 which are complementary to the mating elements 628 and shaped to receive and mate with the mating elements 628. As shown in FIGS. 6 and 8, the mating elements 634 are grooves depressed into the exterior housing 650 of the patient reference sensor 602. Each groove of the two mating elements 634 extends from a bottom of the housing 650 and upward along a height 670 of the patient reference sensor 602 to a point below a top of the housing 650. Each groove is shaped to receive the hook end 630 of either of the two mating elements 628 that creates a tight connection between the two components. Each hook end 630 may snap and lock into place with one of the grooves of the mating elements 634. The grooves of the two mating elements 634 are positioned on opposite exterior sidewalls of the housing 650 from one another, relative to the central axis 610. The two mating elements 634 are symmetrically positioned around the central axis 610 such that the patient reference sensor 602 may be coupled to the mating elements 628 in two different orientations that are 180 degrees rotated from one another. This symmetry feature and 180 degree difference in orientations allows for increased ease of mounting the patient reference sensor 602 to the mounting platform 604 and the ability for the user to rotate the orientation of the patient reference sensor 602 in order to move a positon of the electrical cable 407 during a surgical procedure (e.g., in order to move the cable out of the way).

In order to couple the patient reference sensor 602 with the mounting platform 604, a user may place the central recess 662 over the raised platform 624 in a position such that the mating elements 634 are not aligned with (e.g., may be approximately 90 degrees rotated from) the mating elements 628. A user may then twist (e.g., rotate) the patient reference sensor 602 in a first (e.g., clockwise) direction so that the mating elements 628 lock into place with the mating elements 634. To decouple the patient reference sensor 602 from the mounting platform 604, a user may then twist the patient reference sensor 602 in a second (e.g., counter clockwise) direction, opposite the first direction, to disengage the mating elements 628 from the mating elements 634. When the patient reference sensor 602 is coupled to the mounting platform 604, the bottom surface 660 is in face-sharing contact with the top surface 620.

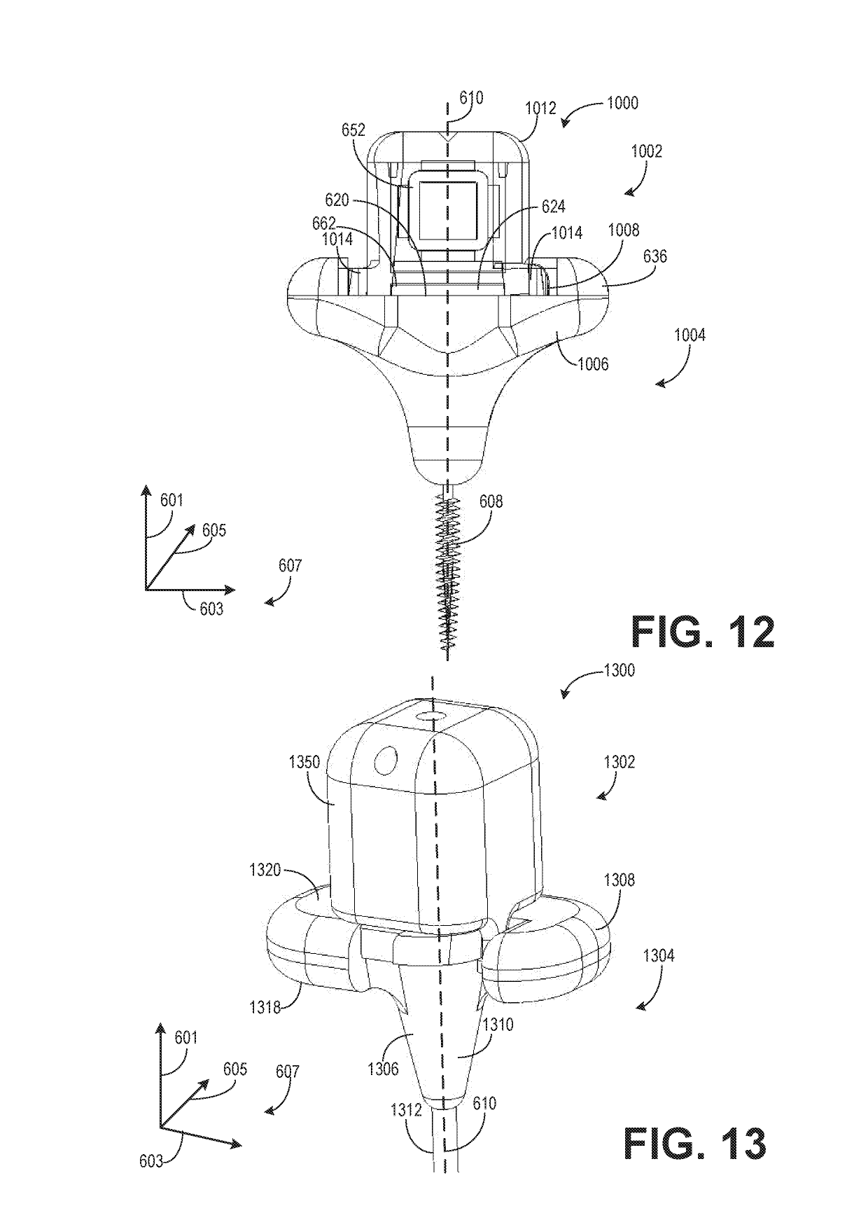

FIGS. 10-12 show the second embodiment of a patient reference assembly 1000 including a patient reference sensor 1002 removably coupled to a mounting platform 1004. FIG. 10 shows an isometric top view of the mounting platform 1004, FIG. 11 shows an isometric view of the patient referencesensor 1002, and FIG. 12 shows a cross-sectional view of the patient reference assembly 1000. The referenceassembly 1000 may be similar to the patient reference assembly 600 described above with reference to FIGS. 6-9. For example, a body 1006 of the mounting platform 1004 may have a similar shape to the body 606 of mounting platform 604, but with differently shaped mating elements 1008 of the mating interface 1010. Thus, similar components between the structures of body 606 and body 1006 have been numbered similarly in FIGS. 10 and 12 and are not re-described below for the sake of brevity. For the mounting platform 1004, the mating elements 1008 are grooves recessed into the rounded ends 636 of the body 1006. As described above, the rounded ends 636 are raised portions of the body 1006 raised above the top surface 620. The grooves of the mating elements 1008 are the same shape but oppositely and symmetrically positioned about the central axis 610. Further, the mating elements 1008 are arranged opposite one another across the raised platform 624.

The r patient reference sensor 1002 includes a similar external housing 1012 to housing 650 of patient reference sensor 602 (e.g., similar in shape and size). Additionally, the internal components of the patient reference sensor 1002 may be the same as the internal components of patient reference sensor 602. However, the mating elements 1014 of the mating interface 1013 of patient reference sensor 1002 are hinges that extend outwardly from and along the housing 1012 at a bottom of the housing 1012. Each of the mating elements 1014 includes a base end 1016 directly coupled to the bottom of the housing 1012 and a free, hook end 1018 shaped to couple with one (and either of) the grooves of the mating elements 1008. The hinges of the mating elements 1014 are the same shape but symmetrically positioned about the central axis 610. Further, the mating elements 1014 are arranged on opposite exterior sidewalls of the housing 1012 from one another. It should be noted that the hinged mating elements 1014 of patient reference sensor 1002 and hinged mating elements 628 of the mounting platform 604 shown in FIGS. 6-7 may have an amount of compliance so they may flex and snap into and out of the corresponding grooved mating elements that they are shaped to mate with. The mating interface 1013 of patient reference sensor 1002 may couple and uncouple from the mating interface 1010 of the mounting platform 1004 in a similar way to that described above for patient reference assembly 600.

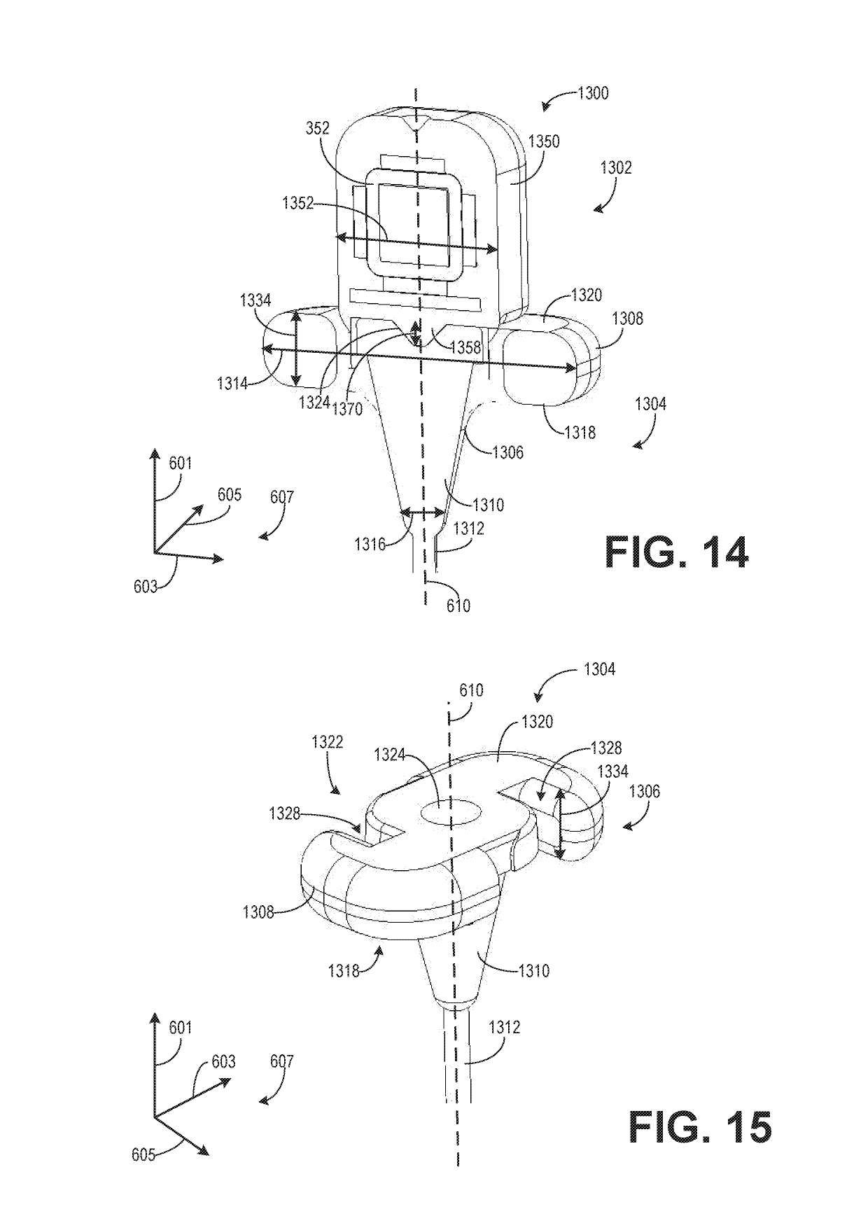

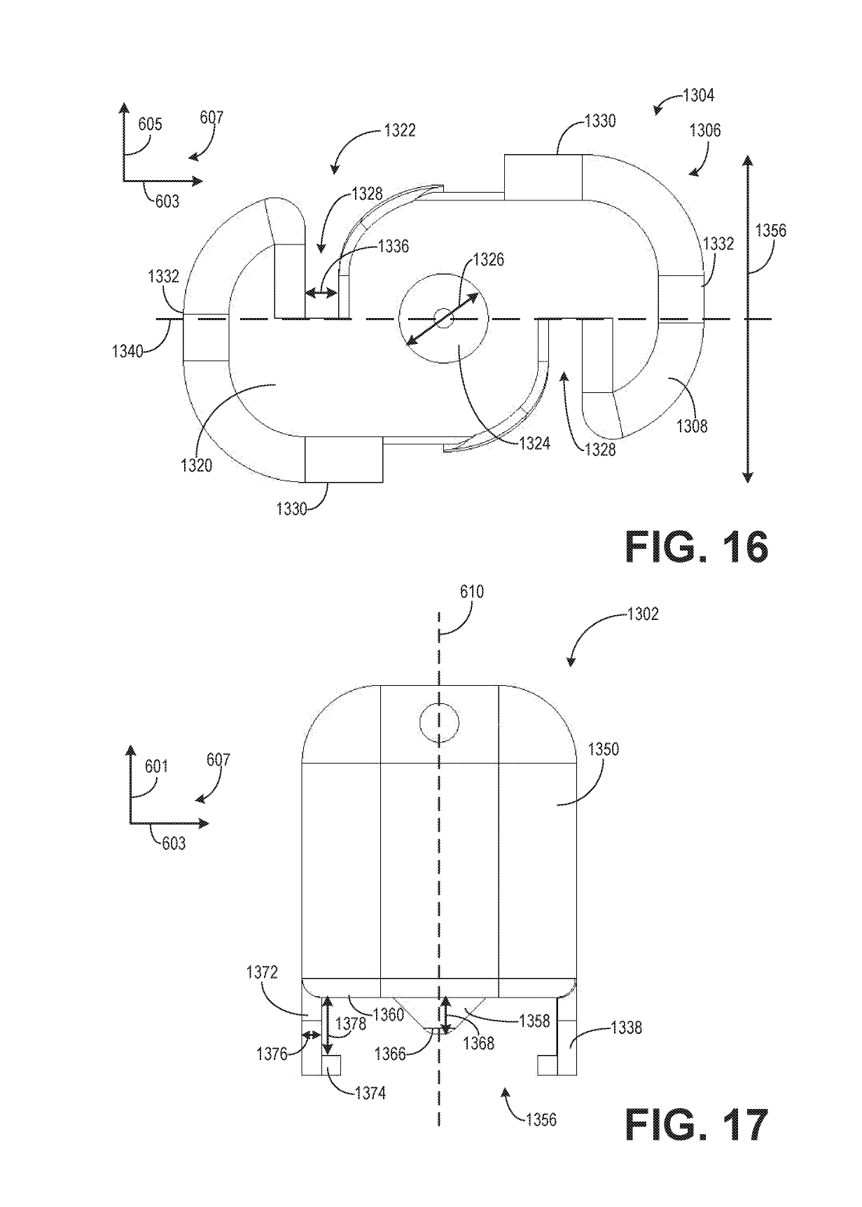

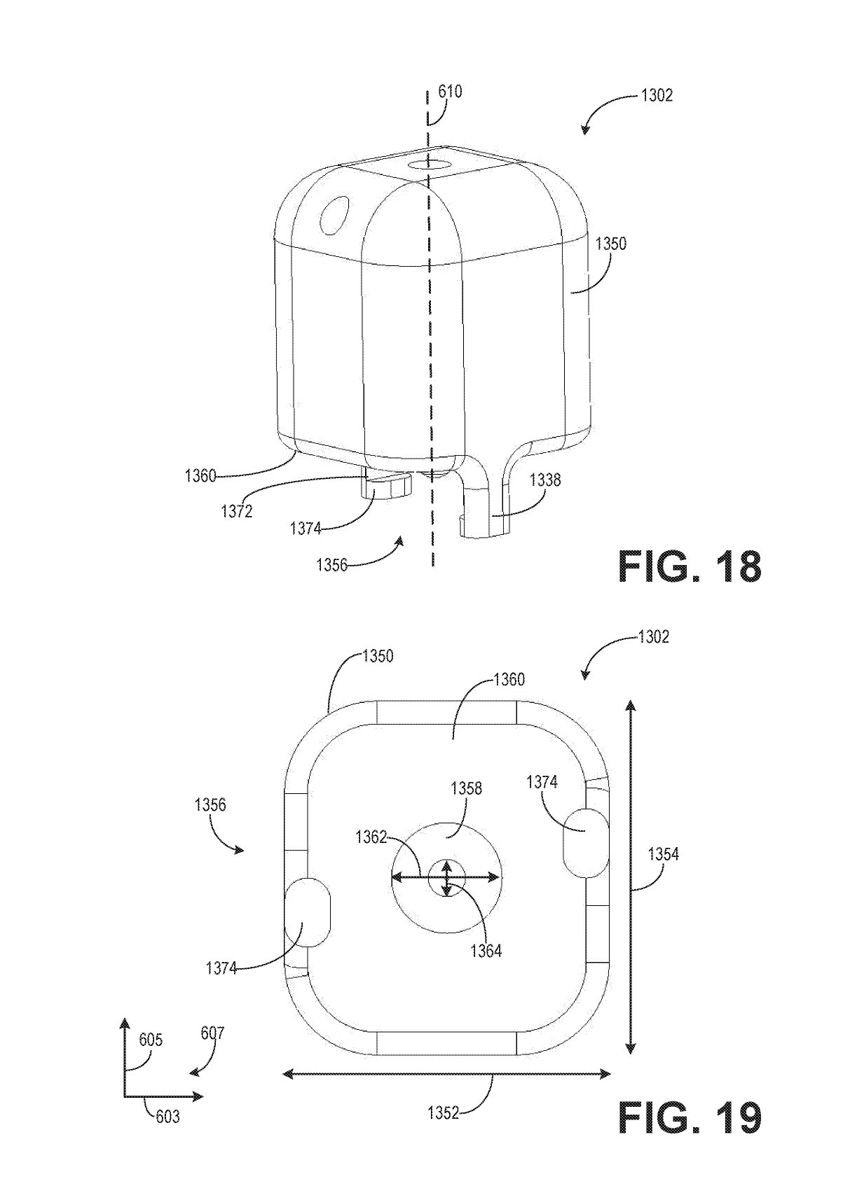

FIGS. 13-19 show the third embodiment of a patient reference assembly 1300 including a patient reference sensor 1302 removably coupled to a mounting platform 1304. FIG. 13 shows an isometric view of the patient reference assembly 1300, FIG. 14 shows a cross-sectional view of the patient reference assembly 1300, FIG. 15 shows an isometric view of the mounting platform 1304, FIG. 16 shows a top view of the mounting platform 1304, FIG. 17 shows a side view of the patient reference sensor 1302, FIG. 18 shows an isometric view of the patient reference sensor 1302, and FIG. 19 shows a bottom view of the patient referencesensor 1302.

Referring to FIGS. 13-16, the mounting platform 1304 includes a body 1306 shaped for attaching (e.g., installing) the mounting platform 1304 on the patient's tissue (e.g., anatomy) without the use of a secondary installation tool. Specifically, the body 1306 is oblong with rounded ends at a top, platform portion 1308 of the mounting platform (relative to the vertical axis 301 and a surface on which a patient to which the mounting platform is coupled sits). The body 1306 then tapers inward (toward the central axis 610) in a downward direction (e.g., negative direction along vertical axis 601) from the platform portion 1308 to a bottom portion 1310, along the central axis 610. The bottom portion 1310 of the body 606, which is closest to an attachment interface 1312, is a narrower part of the body 1306 than the top platform portion 1308 and has a relatively circular cross-section (which tapers as it gets closer to the attachment interface 1312) in a plane of the horizontal axis 603 and lateral axis 605. The body 1306 may not include a handle or protrusion and instead the platform portion 1308 may be gripped by a user when installing and coupling the mounting platform 1304 to the patient. Specifically, the platform portion 1308 may enable a user to screw or insert the attachment interface 1312 into or onto the patient's bone (or other tissue) in order to rigidly fix the mounting platform 1304 to the patient without the use of a secondary attachment tool. In this way, only a user's own force may be used to fix the mounting platform 1304 to the patient.

As seen in FIG. 14, the oblong, platform portion 1308 of the body 1306 has a first length 1314 in a direction of the horizontal axis 603 which is the longest part of the patient reference assembly 1300. The bottom portion 1310 has a second length 1316, which tapers along its height to the attachment interface 1312 and even at its widest is smaller than the first length 1314. The bottom portion 1310 is conical in shape. As shown in FIGS. 13-16, the outer surfaces of the body 1306 have rounded edges, thereby increasing comfort for a user holding and installing the mounting platform 1304.

The attachment interface 1312 is coupled to and extends from a bottom of the bottom portion 1310. As shown in FIGS. 13-15, the attachment interface 1312 is a bone screw or pin; however, in alternate embodiments a different attachment interface (such as a bone clamp, adhesive plate, etc.) may be coupled to a bottom of the bottom portion 1310 of the body 1306. In one example, the body 1306 may be over-molded on the attachment interface 1312. Said another way, the body 1306 may be composed of a plastic material which is molded around the attachment interface 1312 which may be a metal pin or screw (e.g., composed of titanium or stainless steel).

The bottom portion 1310 extends from a bottom surface 1318 of the platform portion 1308. The platform portion 1310 further includes a top surface 1320 arranged opposite the bottom surface 1318. The top surface 1320 is rectangular-shaped with rounded ends. Additionally, the top surface 1320 is planar. The platform portion 1308 forms a first mating interface 1322 which includes a central recess 1324 depressed inward (relative to the vertical axis 601, in the negative direction of the vertical axis) from the planar, top surface 1320. As shown in FIGS. 15 and 16, the central recess 1324 is circular, centered along the central axis 610, and has an outer dimeter 1326 at the top surface 1320 which decreases as the recess depresses further into an interior of the platform portion 1310. In alternate embodiments, the central recess 1324 may have a differently shaped cross-section such as square, triangular, oval, star, or the like.

The first mating interface 1322 further includes a pair of mating elements in the form of recessed slots 1328. The recessed slots 1328 extend inward, toward the central axis 610, from an outer perimeter of the platform portion 1308. Specifically, as shown in FIG. 16, each recessed slot 1328 first carves into the outer perimeter of the platform portion 1308 at a central location on one of the longer sides 1330 (e.g., the platform portion 1308 includes two oppositely positioned longer sides 1330 arranged along the horizontal axis 603 and two oppositely positioned shorter sides 1332 arranged along the lateral axis 605, with curved transitions between the longer and shorter sides) and extends outward (away from the central axis 610) along a portion of the length of the longer side 1330. Each of the recessed slots 1328, near the curved transition between the longer side 1330 and shorter side 1332, then extends inward, toward the central axis 610, into the platform portion 1308, until reaching a horizontal central axis 1340 of the platform portion 1308. Additionally, each of the recessed slots 1328 extends through an entire height 1334 of the platform portion 1308 (as seen in FIGS. 14 and 15).

The recessed slots (e.g., a width 1336 of each recessed slot) 1328 are shaped and sized to mate with complementary mating elements (e.g., mating arms) 1338 on the patient reference sensor 1302, as described further below, when the patient reference sensor 1302 is directly and removably coupled with the mounting platform 1304. As shown in FIG. 16, the two recessed slots 1328 are shaped the same and symmetrically positioned in the platform portion 1308, around the central axis 610. Specifically, each of the two recessed slots 1328 are spaced apart from one another and arranged on opposite sides of the platform portion 1308. For example, a first recessed slot is positioned in a first longer side 1330 and a second recessed slot is positioned in a second longer side 1330, the first and second longer sides 1330 opposite one another across the horizontal central axis 1340. In this way, the two recessed slots 1328 are oppositely arranged and symmetric about the central axis 610.

As shown in FIGS. 13, 14, and 17-19, the patient reference sensor 1302 includes an exterior (e.g., outer) housing 1350 encasing and surrounding internal components of the patient reference sensor 1302 (which may be the same as internal components of the patient reference sensor 602, as described above). As shown in FIGS. 14 and 19, a length 1352 of the patient reference sensor 1302 is shorter than length 1314 of the platform portion 1308 of the body 1306. Additionally, a width 1354 (shown in FIG. 19) of the patient reference sensor 1302 may be approximately the same as a width 1356 (shown in FIG. 16) of the platform portion 1308 of the body 1306.

As seen in FIGS. 17-20, the patient reference sensor 1302 includes a second mating interface 1356 including a central protrusion 1358 centrally positioned along the central axis 610 and extending outward from a bottom surface 1360 of the patient reference sensor 1302. The central protrusion 1358 is conical in shape and includes a base diameter 1362 arranged at the bottom surface 1360. The base diameter 1362 may be slightly smaller than diameter 1326 of the central recess 1324 so that the central recess 1324 may receive and fit around (and mate with) the central protrusion 1358 (as seen in FIG. 14). The central protrusion 1358 includes an apex 1366 with an apex diameter 1364 that is smaller than the base diameter 1362. The central protrusion tapers in diameter from a base of the central protrusion 1358 arranged at the bottom surface 1360 to the apex 1366 arranged away from the bottom surface 1360. The central protrusion 1358 has a height 1368 which is approximately the same as a depth 1370 of the central recess 1324 (as seen in FIG. 14).

The second mating interface 1356 also includes a pair of mating elements in the form of mating arms 1338. The mating arms 1338 are complementary to the recessed slots 1328 and are shaped to fit within and mate with the recessed slots 1328. As shown in FIGS. 17 and 18, each of the mating arms 1338 includes an arm portion 1372 coupled to an end portion 1374. The arm portion 1372 extends downward and away from the bottom surface 1360 of the housing 1350, at an outer perimeter of the housing 1350. The end portion 1374 extends inward toward the central axis 610 from the arm portion 1372. A width 1376 of the arm portion 1372 is slightly smaller than the width 1336 of the recessed slot 1328 so that the arm portion 1372 fits within the recessed slot 1328. A height 1378 of the arm portion 1372 is approximately the same as the height 1334 of the platform portion 1308. As a result, the end portion 1374 of each mating arm 1338 extends under the bottom surface 1318 of the platform portion 1308 so that the end portion 1374 may be in face-sharing contact with a portion of the bottom surface 1318, thereby securely holding the patient reference sensor 1302 in place with the mounting platform 1304.

Each mating arm 1338 extends into and through each recessed slot 1328. Each mating arm 1338 may snap and lock into place with one of the recessed slots 1328. Additionally, each mating arm 1338 is positioned on opposite exterior sidewalls of the housing 1350 relative to the central axis 610. The two mating arms 1338 are symmetrically positioned around the central axis 610 such that the patient reference sensor 1302 may be coupled to the mounting platform 1304 in two different orientations that are 180 degrees rotated from one another. This symmetry feature and 180 degree difference in orientations allows for increased ease of mounting the patient reference sensor 1302 to the mounting platform 1304 and the ability for the user to rotate the orientation of the patient reference sensor 1302 in order to move a positon of an electrical cable coupled to the patient reference sensor 1302 during a surgical procedure (e.g., in order to move the cable out of the way). Further, it should be noted that when the patient reference sensor 1302 is coupled with the mounting platform 1304 via the first and second mating elements, the bottom surface 1360 of the patient reference sensor 1302 is in face-sharing contact with the top surface 1320 of the mounting platform 1304. This may also be true for the other patient reference assembly embodiments described herein.

In order to couple the patient reference sensor 1302 with the mounting platform 1304, a user may place the central protrusion 1358 over the central recess 1324 in a position such that the mating arms 1338 are not aligned with (e.g., may be approximately 90 degrees rotated from) interior portions of the recessed slots 1328. For example, the mating arms 1338 may be positioned at an edge of the recessed slots 1328 on the longer sides 1330 of the platform portion 1308. A user may then twist (e.g., rotate) the patient reference sensor 1302 in a first direction so that the mating arms 1338 lock into place with the recessed slots 1328. To decouple the patient reference sensor 1302 from the mounting platform 1304, a user may then twist the patient reference sensor 1302 in a second direction, opposite the first direction, to disengage the mating arms 1338 from the recessed slots 1328. Due to the symmetric nature of the recessed slots 1328 and mating arms 1338, each mating arm may be coupled with each and either of the recessed slots 1328.

FIG. 20 shows an embodiment of a mounting platform 2000 of a patient reference assembly including a handle 2002. The mounting platform 2000 is similar to the mounting platform 604 shown in FIG. 7 and has a similar mating interface adapted to mate with a patient reference sensor, such as sensor 602 shown in FIG. 6. However, the mounting platform 2000 may take the shape of any of the mounting platforms described herein and is purely illustrative of how the handle 2002 may be included in a body 2004 of the mounting platform 2000. For example, the handle 2002 may be in place of the protrusion 612 shown in FIG. 7 (e.g., the handle 2002 is longer than the protrusion 612). As such, any of the mounting platforms described herein may include the handle 2002 for enabling a user to mount the mounting platform to the patient. As shown in FIG. 20, the handle 2002 extends outwardly from a central portion of the body 2004, away from the central axis 610, on one side of the body 2004. A width of the handle 2002 may taper inward from the length of the top surface 2006 of the body 2004 to a tip of the handle 2002. The external surfaces of the handle 2002 may be rounded and a length of the handle 2002 may be long enough for a user's hand to grip around the handle 2002.

FIG. 21 shows another embodiment of a mounting platform 2100 of a patient reference assembly including a wheel-shaped handle 2102. The mounting platform 2100 is similar to the mounting platform 604 shown in FIG. 7 and has a similar mating interface adapted to mate with a patient reference sensor, such as patient reference sensor 603 shown in FIG. 6. However, the mounting platform 2100 may take the shape of any of the mounting platforms described herein and is purely illustrative of how the wheel-shaped handle 2102 may be included in a body 2104 of the mounting platform 2100. For example, the wheel-shaped handle 2102 may be in place of the protrusion 612 shown in FIG. 7. As such, any of the mounting platforms described herein may include the handle 2102 for enabling a user to mount the mounting platform to the patient. As shown in FIG. 21, the wheel-shaped handle 2102 is integrally formed with the body 2104. The wheel-shaped handle 2102 includes a plurality of rounded protrusions 2106 spaced apart from one another about the central axis 610. For example, the protrusions 2106 may be equally spaced around an outer perimeter of the wheel-shaped handle 2102, about the central axis 610. In this way, the wheel-shaped handle 2102 may resemble a knob. As shown in FIG. 21, the wheel-shaped handle 2102 includes eight protrusions 2106; however, a different number of protrusions (greater or less than eight) are possible.

FIG. 22 shows an embodiment of a mounting platform 2204 of a patient reference assembly 2200 including a surface mount 2206. As shown in FIG. 22, the surface mount is in the form of a plate. A patient reference sensor 2202 may take a form of any of the patient reference sensors described herein with any of the above-described mating interfaces. The mounting platform 2204 may then include a complementary mating interface to that of the patient reference sensor 2202 such that the two components may be removably coupled to one another. However, instead of the mounting platforms already described above with reference to the other embodiments, the mounting platform 2204 may include the surface mount 2206. The mating interface 2208 of the mounting platform 2204 is positioned on a top surface of the surface mount 2206. A bottom surface of the surface mount 2206 may include an adhesive element for adhering the mounting platform 2204 to a patient (e.g., such as adhering the mounting platform 2204 to a patient's skin). As shown in FIG. 22, the surface mount is a circular plate; however, in alternate embodiment the surface mount 2206 may have a different shape (e.g., such as square, rectangular, oval, etc.). Additionally, the surface mount 2206 may be flexible so that it may more easily fit to contours of a patient's skin.

FIG. 23 shows an embodiment of a mounting platform 2304 of a patient reference assembly 2300 including a clamp 2306 as the attachment interface 2308. A patient reference sensor 2302 may take a form of any of the patient reference sensors described herein with any of the above-described mating interfaces. The mounting platform 2304 may then include a complementary mating interface to that of the patient reference sensor 2302 such that the two components may be removably coupled to one another. However, instead of a bone pin or screw, the mounting platform 2304 includes an attachment interface 2308 in the form of a clamp 2306. The clamp 2306 extends from a bottom of a body 2310 of the mounting platform 2304. In one example, the clamp 2306 may be a bone clamp with two clamping arms shaped to clamp against a bone, such as a spinous process. The clamp 2306 may include a latching feature 2312 that allows a user to couple and decouple the clamp 2306 from the patient's bone or other tissue without the use of additional attachment tools.

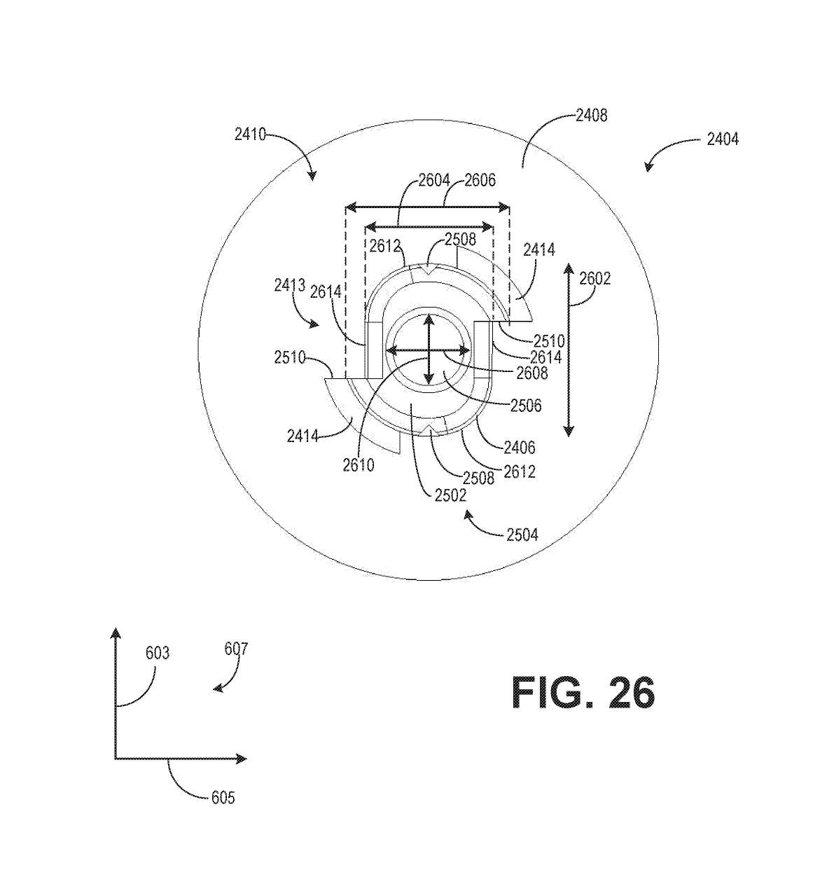

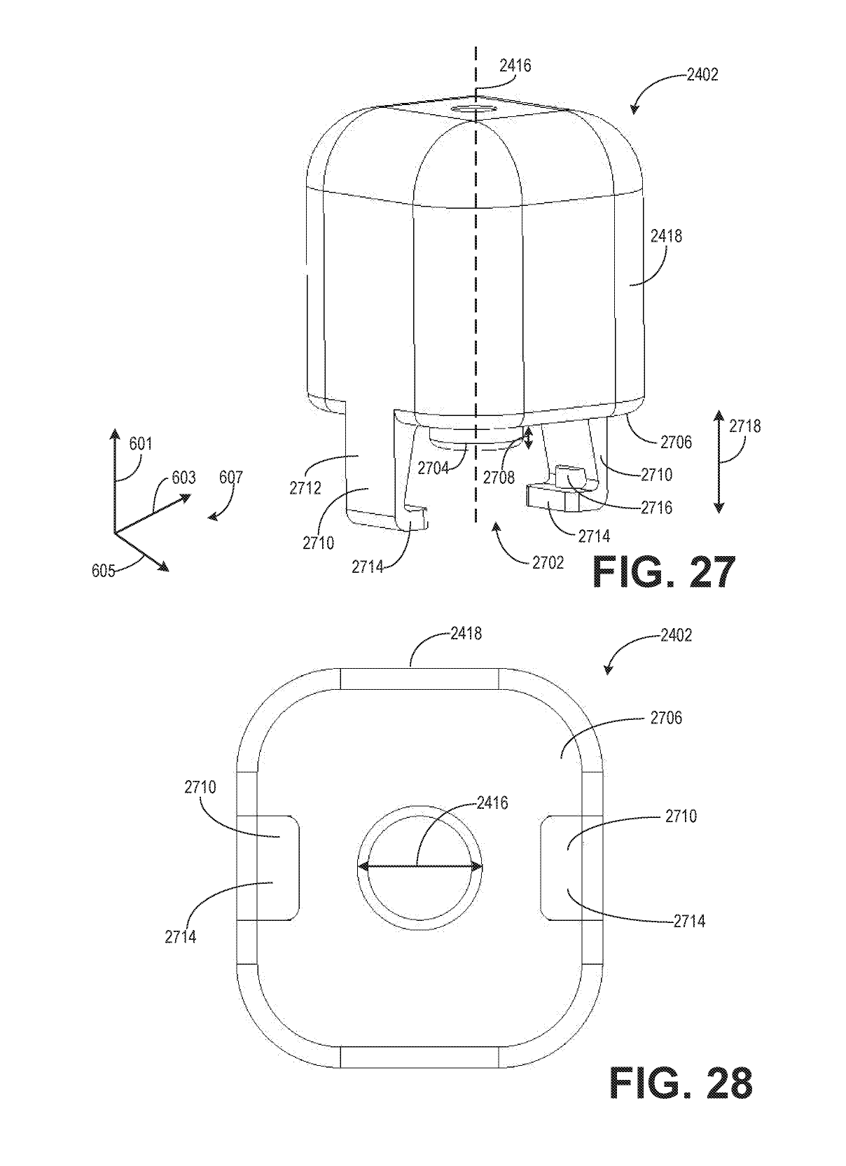

FIGS. 24-28 show the fourth embodiment of a patient reference assembly 2400 including a patient reference sensor 2402 removably coupled to a mounting platform 2404. FIG. 24 shows an isometric view of the patient reference assembly 2400, FIG. 25 shows an isometric view of the mounting platform 2404 of the patient reference assembly 2400, FIG. 26 shows a top view of the mounting platform 2404 of the patient reference assembly 2400, FIG. 27 shows an isometric view of the patient reference sensor 2402, and FIG. 28 shows a bottom view of the patient reference sensor 2402.

Referring to FIGS. 24-26, the mounting platform 2404 includes a body 2406 shaped for receiving the patient reference sensor 2402 and an attachment interface 2408 shaped for attaching (e.g., installing) the mounting platform 2404 on the patient's tissue (e.g., anatomy) without the use of a secondary installation tool. As shown in FIGS. 24-26, the attachment interface 2408 is a surface mount (similar to surface mount 2206 shown in FIG. 22) in a shape of a circular plate. However, in alternate embodiments, the attachment interface 2408 may have a different cross-sectional shape such as square, triangular, oblong, or the like. A bottom surface 2409 of the attachment interface 2408 may include an adhesive for adhering the attachment interface 2408 directly to the skin of the patient. A top surface 2410 of the attachment interface 2408 is directly coupled to the body 2406 of the mounting platform 2404. In alternate embodiments, the body 2406 may be coupled to a different type of attachment interface, such as one of the attachment interfaces shown in FIGS. 13-16 or FIG. 23.

As shown in FIGS. 24-26, the body 2406 is oblong with rounded ends. The body 2406 extends upward and outward from the top surface 2410 of the attachment interface 2408. Thus, the body 2406 has a height 2412 defined between a base of the body 2406 arranged at the top surface 2410 and a top surface 2502 of the body 2406 (where the top surface is opposite the bottom surface 2408 of the attachment interface 2408 which is adapted to attach directly to the patient). The body 2406 does not include a handle or protrusion and instead the body 2406 may be gripped by a user when installing and coupling the mounting platform 2404 to the patient. Specifically, the body 2406 may enable a user to adhere the attachment interface to a patient's skin surface (or screw or insert the attachment interface into or onto the patient's bone if the attachment interface is a bone screw or clamp). Thus, the user may rigidly fix the mounting platform 2404 to the patient without the use of a secondary attachment tool. In this way, only a user's own force may be used to fix the mounting platform 2404 to the patient.

The body 2406 includes a platform portion 2413 and feet portions 2414 which taper downward and outward (relative to a central axis 2416 of the patient reference assembly 2400) from the platform portion 2413 to the top surface 2410 of the attachment interface 2408. The feet portions 2414 directly couple the platform portion 2413 to the top surface 2410. In one example, the body feet portions 2414 may be over-molded on the attachment interface 2408. Specifically, as shown in FIG. 25, the platform portion 2413 is raised above the top surface 2410 by the feet 2414, thereby forming at space (e.g., void) 2415 between the top surface 2410 and a bottom surface 2417 of the platform portion 2413, the bottom surface 2417 opposite the top surface 2502.

As seen in FIG. 26, the oblong, platform portion 2413 of the body 2406 has a length 2602 which is the longest part of the platform portion 2413, a first width 2604, and a second width 2606 which is smaller than the first width 2604. The top surface 2502 of the body is also a top surface of the platform portion 2413. The top surface 2502 is rectangular-shaped with curved (e.g., semi-circular) ends. Additionally, the top surface 2502 is relatively planar.

As shown in FIGS. 25 and 26, the platform portion 2413 forms a first mating interface 2504 which includes a central recess 2506 depressed inward (relative to the vertical axis 601, in the negative direction of the vertical axis) from the planar, top surface 2502. As shown in FIGS. 25 and 26, the central recess 2506 is circular and centered along the central axis 2416. Additionally, as shown in FIG. 26, the central recess 2506 has an outer diameter 2608 at the top surface 2502 and an inner diameter 2610 at the bottom of the central recess 2506. The inner diameter 2610 may be a recess diameter of a majority of the recess 2506 and a top portion (e.g., at a lip of the recess) of the recess 2506 is curved between the outer diameter 2608 and inner diameter 2610. In alternate embodiments, the central recess 2506 may have a differently shaped cross-section such as square, triangular, oval, star, or the like. Further, the central recess 2506 is depressed into an interior of the platform portion 2413, toward the top surface 2410 of the attachment interface 2408, at a depth.

The first mating interface 2504 further includes two first mating elements in the form of recessed slots 2508. The recessed slots 2508 extend inward, toward the central axis 2416, from an outer perimeter of the platform portion 2413. Specifically, as shown in FIGS. 25 and 26, each recessed slot 2508 depressed into the outer perimeter of the platform portion 2413 at a central location on one of the curved sides 2612 (e.g., the platform portion 2413 includes two oppositely positioned straight sides 2614 arranged along the horizontal axis 603 and two oppositely positioned curved (semi-circular) sides 2612 arranged along the lateral axis 605). Additionally, each of the recessed slots 2508 extends through an entire height of the platform portion 2413 (which is shorter than the height 2412 of the entire body 2406).

The recessed slots 2508 are shaped and sized to mate with complementary mating elements on the patient reference sensor 2402, as described further below, when the patient reference sensor 2402 is directly and removably coupled with the mounting platform 2413. As shown in FIGS. 25 and 26, the recessed slots 2508 have a triangular cross-section. However, in alternate embodiments, a differently shaped cross-section is possible (e.g., square, rectangular, semi-circular, circular, or the like). Additionally, the two recessed slots 2508 are shaped the same and symmetrically positioned in the platform portion 2413, around the central axis 2416. Specifically, each of the two recessed slots 2508 are spaced apart from one another and arranged on opposite sides of the platform portion 2413. For example, a first recessed slot is positioned in a first curved side 2612 and a second recessed slot is positioned in a second curved side 2612, the first and second curved sides 2612 opposite one another across the horizontal central axis 2416. In this way, the two recessed slots 2508 are oppositely arranged and symmetric about the central axis 2416.

Additionally, the platform portion 2413 includes oppositely facing edges 2510 which are part of the curved sides 2612 and extend outward from the straight sides 2614 and central axis 2416.

As shown in FIGS. 24, 27, and 28, the patient reference sensor 2402 includes an exterior (e.g., outer) housing 2418 encasing and surrounding internal components of the patient reference sensor 2402 (which may be the same as internal components of the patient reference sensor 602, as described above). The patient reference sensor 2402 includes a second mating interface 2702 including a central protrusion 2704 centrally positioned along the central axis 2416 and extending outward from a bottom surface 2706 of the patient reference sensor 2402. The central protrusion 2704 is cylindrical in shape and has a diameter 2802. An end of the central protrusion 2704 is chamfered so that the end may have a slightly smaller diameter than diameter 2802. The diameter 2802 may be slightly smaller than diameter 2610 of the central recess 2506 so that the central recess 2506 may receive and fit around (and mate with) the central protrusion 2704. The central protrusion 2704 has a height 2708 which is approximately the same as a depth f the central recess 2506.

The second mating interface 2702 also includes a pair of mating elements in the form of mating arms 2710. As shown in FIG. 27, each of the mating arms 2710 includes an arm portion 2712 coupled to an end portion 2714. The arm portion 2712 extends downward and away from the bottom surface 2706 of the housing 2418, at an outer perimeter of the housing 2418. The end portion 2714 extends inward toward the central axis 2416 from the arm portion 2712. Each of the mating arms 2710 includes a protrusion 2716 at the end portion 2714 and extending inward toward the central axis 2416 from an inner surface of the arm portion 2712. The protrusions 2716 of the mating arms 2710 are complementary to the recessed slots 2508 and are shaped to fit within and mate with the recessed slots 2508. In one example, a cross-section of the protrusions 2716 is semi-circular and fits within the triangular cross-section of the recessed slots 2508, thereby allowing some play in the mating connection to account for variabilities in machining of the components. In another example, the cross-section of the protrusions 2716 may be triangular and fits within the triangular cross-section of the recessed slots 2508.

A height 2718 of the arm portion 2712 is approximately the same as a height of the platform portion 2413. As a result, the end portion 2714 of each mating arm 2710 extends under the bottom surface 2417 of the platform portion 2413 so that the end portion 2714 may be in face-sharing contact with a portion of the bottom surface 2417, thereby securely holding the patient reference sensor 2402 in place with the mounting platform 2404.

Each mating arm 2710 may snap and lock into place with one of the recessed slots 2508. Additionally, each mating arm 2710 is positioned on opposite exterior sidewalls of the housing 2418 relative to the central axis 2416. The two mating arms 2710 are symmetrically positioned around the central axis 2416 such that the patient reference sensor 2402 may be coupled to the mounting platform 2404 in two different orientations that are 180 degrees rotated from one another. This symmetry feature and 180 degree difference in orientations allows for increased ease of mounting the patient reference sensor 2402 to the mounting platform 2404 and the ability for the user to rotate the orientation of the patient reference sensor 2402 in order to move a positon of an electrical cable coupled to the patient reference sensor 2402 during a surgical procedure (e.g., in order to move the cable out of the way). Further, it should be noted that when the patient reference sensor 2402 is coupled with the mounting platform 2404 via the first and second mating elements, the bottom surface 2706 of the patient reference sensor 2402 is in face-sharing contact with the top surface 2502 of the mounting platform 2404.

In order to couple the patient reference sensor 2402 with the mounting platform 2404, a user may place the central protrusion 2704 over the central recess 2506 in a position such that the mating arms 2710 are not aligned with (e.g., may be approximately 90 degrees rotated from) the recessed slots 2508. For example, the mating arms 2710 may be positioned at the straight sides 2614 of the platform portion 2413. A user may then twist (e.g., rotate) the patient reference sensor 2402 in a first direction so that the mating arms 2710 lock into place with the recessed slots 2508. To decouple the patient reference sensor 2402 from the mounting platform 2404, a user may then twist the patient reference sensor 2402 in a second direction, opposite the first direction, to disengage the mating arms 2710 from the recessed slots 2508. Due to the symmetric nature of the recessed slots 2508 and mating arms 2710, each mating arm may be coupled with each and either of the recessed slots 2508.

In each of the above embodiments of the patient reference assembly, the components, including the mounting platform, may be manufactured via an injection molding process. This may increase the ease of manufacturing the components of the patient reference assembly without having trap zones and still having adequate draft. Further, the components may be manufactured in single pull direction via the injection molding process.

In this way, a patient reference assembly for an electromagnetic surgical navigation system may include a sensor (e.g., patient reference sensor that is one of a transmitter or receiver) removably coupled to a mounting platform via complementary mating interfaces of the patient reference sensor and mounting platform. The complementary mating interfaces may take different forms, as described here, but may including mating elements that interlock the sensor and mounting platform to one another, thereby forming a tight connection between the outer housing of the sensor and the mounting platform. The mating interfaces are symmetrically arranged such that the sensor may be orientated in two different positions on the mounting interface. This increases the ease of installation, as well as providing for better cable management (e.g., moving the electrical cable of the sensor out of the way) during a surgical or other medical procedure. In one example, multiple mounting platforms may be positioned on the patient prior to a surgery. During a surgical procedure, the same patient reference sensor may be moved around to different mounting platforms depending on where a user is operating. Further, the different mounting platforms may have the same mating interface but may have different attachment interfaces. Additionally, the patient reference sensor is in face-sharing contact with and positioned on top of the mounting platform. This, along with the shapes of the body of the mounting platform, provides a slim design and compact size for the entire assembly, thereby reducing collisions with other objects during a surgical procedure and thus the need to re-register navigation images. Further still, an ergonomic shape of the body of the mounting platform may increase the ease of installing (e.g., attaching) the mounting platform to a patient without the use of secondary attachment tools, thereby saving time and component costs. Thus, a technical effect of having a patient reference sensor and mounting platform with complementary and symmetric mating interfaces that removably couple to one another is having an assembly that simplifies the process of installing the assembly, increases ease of use during a surgical procedure, and reduces collisions with the assembly that may require recalibration or re-registration of images.

In one embodiment, a patient reference assembly for an electromagnetic surgical navigation system comprises: a mounting platform including a first mating interface and an attachment interface shaped to couple to a patient; and a sensor including a second mating interface shaped to removably couple with the first mating interface. In a first example of the patient reference assembly for the electromagnetic surgical navigation system, the first mating interface includes two first mating elements symmetrically positioned relative to a central axis of the patient reference assembly and the second mating interface includes two second mating elements symmetrically positioned relative to the central axis and wherein each of the two first mating elements is shaped to couple with each of the two second mating elements. A second example of the patient reference assembly for the electromagnetic surgical navigation system optionally includes the first example and further includes wherein the sensor includes an outer housing including the second mating interface and one or more electromagnetic coils positioned within an interior of the outer housing. A third example of the patient reference assembly for the electromagnetic surgical navigation system optionally includes one or more of the first and second examples, and further includes wherein the attachment interface includes one of a screw, a surface mount including an adhesive, and a clamp. A fourth example of the patient reference assembly for the electromagnetic surgical navigation system optionally includes one or more of the first through third examples, a further includes wherein when the sensor is removably coupled with the mounting platform via the first and second mating interfaces, a bottom surface of an outer housing of the sensor is in face-sharing contact with a top surface of a body of the mounting platform. A fifth example of the patient reference assembly for the electromagnetic surgical navigation system optionally includes one or more of the first through fourth examples, a further includes wherein the first mating interface includes a central recess in a top surface of a platform portion of the mounting platform and a pair of recessed slots in the platform portion and wherein the attachment interface extends from a bottom surface of the platform portion. A sixth example of the patient reference assembly for the electromagnetic surgical navigation system optionally includes one or more of the first through fifth examples, a further includes wherein the sensor includes an exterior housing and one or more electromagnetic coils positioned within the housing and wherein the second mating interface includes a central protrusion and a pair of mating arms extending from a bottom surface of the housing. A seventh example of the patient reference assembly for the electromagnetic surgical navigation system optionally includes one or more of the first through sixth examples, a further includes wherein each mating arm of the pair of mating arms includes a protrusion extending from an inner surface of the mating surface which is shaped to mate with one recessed slot of the pair of recessed slots. An eighth example of the patient reference assembly for the electromagnetic surgical navigation system optionally includes one or more of the first through seventh examples, a further includes wherein the first mating interface includes a central raised platform and a pair of hinges spaced apart from and arranged on opposite sides of the raised platform and wherein the second mating interface includes a central depression shaped to fit around and mate with the raised platform and a pair of grooves positioned on exterior sidewalls of a housing of the sensor, the pair of grooves shaped to receive and mate with the pair of hinges. A ninth example of the patient reference assembly for the electromagnetic surgical navigation system optionally includes one or more of the first through eighth examples, a further includes wherein the first mating interface includes a central raised platform and a pair of grooves spaced apart from and arranged on opposite sides of the raised platform and wherein the second mating interface includes a central depression shaped to fit around and mate with the raised platform and a pair of hinges positioned on exterior sidewalls of a housing of the sensor, the pair of hinges shaped to interlock with the pair of grooves. A tenth example of the patient reference assembly for the electromagnetic surgical navigation system optionally includes one or more of the first through ninth examples, a further includes wherein the mounting platform includes a body shaped for attaching the attachment interface to the patient, where the first mating surface is positioned at a top surface of the body and the attachment interface is positioned at a bottom surface of the body, the bottom surface opposite the top surface. An eleventh example of the patient reference assembly for the electromagnetic surgical navigation system optionally includes one or more of the first through tenth examples, a further includes wherein the body includes one of a single protrusion extending outward from the body and a handle extending outward from the body. A twelfth example of the patient reference assembly for the electromagnetic surgical navigation system optionally includes one or more of the first through eleventh examples, a further includes wherein the body includes a wheel-shaped handle integrally formed with a remainder of the body, the wheel-shaped handle including a plurality of rounded protrusions spaced apart from one another about a central axis of the transmitter assembly.

In another embodiment, an electromagnetic surgical navigation system comprises: a patient reference assembly including a first mounting platform and a patient reference sensor including a first electromagnetic coil, the patient reference sensor removably coupled to a top surface of the first mounting platform via a first interlocking interface of the first mounting platform and a second interlocking interface of the patient reference sensor, where the first mounting platform includes a first attachment interface shaped to couple to patient and extending from a bottom surface of the first mounting platform; and a medical instrument tracking assembly not coupled to the patient and including a second electromagnetic coil, where the second electromagnetic coil is adapted to sense an electromagnetic field produced by the first electromagnetic coil. In a first example of the electromagnetic surgical navigation system, the system further comprises a second mounting platform having a same first interlocking interface as the first mounting platform and wherein the second electromagnetic coil is adapted to sense the electromagnetic field produced by the first electromagnetic coil when removably coupled to each of the first mounting platform and the second mounting platform. A second example of the electromagnetic surgical navigation system optionally includes the first example and further includes wherein the second mounting platform includes a second attachment interface different than the first attachment interface and shaped to couple to the patient in an alternate location than the first attachment interface. A third example of the electromagnetic surgical navigation system optionally includes one or more of the first and second examples, and further includes wherein the patient reference sensor is a transmitter and the second electromagnetic coil of the medical tracking assembly is a receiver. A fourth example of the electromagnetic surgical navigation system optionally includes one or more of the first through third examples, and further includes, wherein the patient reference sensor is a receiver and the second electromagnetic coil of the medical tracking assembly is a transmitter.

In yet another embodiment, a method for installing a patient reference assembly for an electromagnetic surgical navigation system comprises securing a mounting platform of the patient reference assembly to a patient via directly coupling an attachment interface arranged at a bottom of the mounting platform to the patient without using a secondary attachment tool; and coupling a sensor of the patient reference assembly to a top of the mounting platform via engaging a second interlocking interface on the sensor with a first interlocking interface on the mounting platform via a twist and lock motion. In a first example of the method, the sensor is arranged in a first orientation relative to the mounting platform after the coupling and the method may further comprise decoupling the sensor from the mounting platform without moving the mounting platform and recoupling the sensor to the mounting platform in a second orientation relative to the mounting platform, the second orientation 180 degrees different than the first orientation.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A patient reference assembly for an electromagnetic surgical navigation system, comprising:
a mounting platform including a first mating interface and an attachment interface shaped to couple to a patient; and
a sensor including a second mating interface shaped to removably couple with the first mating interface.

2. The patient reference assembly of claim 1, wherein the first mating interface includes two first mating elements symmetrically positioned relative to a central axis of the patient reference assembly and the second mating interface includes two second mating elements symmetrically positioned relative to the central axis and wherein each of the two first mating elements is shaped to couple with each of the two second mating elements.

3. The patient reference assembly of claim 1, wherein the sensor includes an outer housing including the second mating interface and one or more electromagnetic coils positioned within an interior of the outer housing.

4. The patient reference assembly of claim 1, wherein the attachment interface includes one of a screw, a surface mount including an adhesive, and a clamp.

5. The patient reference assembly of claim 1, wherein when the sensor is removably coupled with the mounting platform via the first and second mating interfaces, a bottom surface of an outer housing of the sensor is in face-sharing contact with a top surface of a body of the mounting platform.

6. The patient reference assembly of claim 1, wherein the first mating interface includes a central recess in a top surface of a platform portion of the mounting platform and a pair of recessed slots in the platform portion and wherein the attachment interface extends from a bottom surface of the platform portion.

7. The patient reference assembly of claim 6, wherein the sensor includes an exterior housing and one or more electromagnetic coils positioned within the housing and wherein the second mating interface includes a central protrusion and a pair of mating arms extending from a bottom surface of the housing.

8. The patient reference assembly of claim 7, wherein each mating arm of the pair of mating arms includes a protrusion extending from an inner surface of the mating surface which is shaped to mate with one recessed slot of the pair of recessed slots.

9. The patient reference assembly of claim 1, wherein the first mating interface includes a central raised platform and a pair of hinges spaced apart from and arranged on opposite sides of the raised platform and wherein the second mating interface includes a central depression shaped to fit around and mate with the raised platform and a pair of grooves positioned on exterior sidewalls of a housing of the sensor, the pair of grooves shaped to receive and mate with the pair of hinges.

10. The patient reference assembly of claim 1, wherein the first mating interface includes a central raised platform and a pair of grooves spaced apart from and arranged on opposite sides of the raised platform and wherein the second mating interface includes a central depression shaped to fit around and mate with the raised platform and a pair of hinges positioned on exterior sidewalls of a housing of the sensor, the pair of hinges shaped to interlock with the pair of grooves.

11. The patient reference assembly of claim 1, wherein the mounting platform includes a body shaped for attaching the attachment interface to the patient, where the first mating surface is positioned at a top surface of the body and the attachment interface is positioned at a bottom surface of the body, the bottom surface opposite the top surface.

12. The patient reference assembly of claim 10, wherein the body includes one of a single protrusion extending outward from the body and a handle extending outward from the body.

13. The patient reference assembly of claim 10, wherein the body includes a wheel-shaped handle integrally formed with a remainder of the body, the wheel-shaped handle including a plurality of rounded protrusions spaced apart from one another about a central axis of the transmitter assembly.

14. An electromagnetic surgical navigation system, comprising:
a patient reference assembly including a first mounting platform and a patient reference sensor including a first electromagnetic coil, the patient reference sensor removably coupled to a top surface of the first mounting platform via a first interlocking interface of the first mounting platform and a second interlocking interface of the patient reference sensor, where the first mounting platform includes a first attachment interface shaped to couple to patient and extending from a bottom surface of the first mounting platform; and
a medical instrument tracking assembly not coupled to the patient and including a second electromagnetic coil, where the second electromagnetic coil is adapted to sense an electromagnetic field produced by the first electromagnetic coil.

15. The electromagnetic surgical navigation system of claim 14, further comprising a second mounting platform having a same first interlocking interface as the first mounting platform and wherein the second electromagnetic coil is adapted to sense the electromagnetic field produced by the first electromagnetic coil when removably coupled to each of the first mounting platform and the second mounting platform.

16. The electromagnetic surgical navigation system of claim 15, wherein the second mounting platform includes a second attachment interface different than the first attachment interface and shaped to couple to the patient in an alternate location than the first attachment interface.

17. The electromagnetic surgical navigation system of claim 14, wherein the patient reference sensor is a transmitter and the second electromagnetic coil of the medical tracking assembly is a receiver.

18. The electromagnetic surgical navigation system of claim 14, wherein the patient reference sensor is a receiver and the second electromagnetic coil of the medical tracking assembly is a transmitter.

19. A method for installing a patient reference assembly for an electromagnetic surgical navigation system, comprising:
securing a mounting platform of the patient reference assembly to a patient via directly coupling an attachment interface arranged at a bottom of the mounting platform to the patient without using a secondary attachment tool; and
coupling a sensor of the patient reference assembly to a top of the mounting platform via engaging a second interlocking interface on the sensor with a first interlocking interface on the mounting platform via a twist and lock motion.

20. The method of claim 19, wherein the sensor is arranged in a first orientation relative to the mounting platform after the coupling and further comprising decoupling the sensor from the mounting platform without moving the mounting platform and recoupling the sensor to the mounting platform in a second orientation relative to the mounting platform, the second orientation 180 degrees different than the first orientation.
